SEQUENCE LISTING (297 sequences)

SEQ ID NO: 1 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
catttccaacttgtagggtt

SEQ ID NO: 2 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtgtttgagaggagccattt

SEQ ID NO: 3 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agtgtttgagaggagccatt

SEQ ID NO: 4 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtagcagatacatttgggct

SEQ ID NO: 5 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agtagcagatacatttgggc

SEQ ID NO: 6 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttttgcattatggaaggcac

SEQ ID NO: 7 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gttttgcattatggaaggca

SEQ ID NO: 8 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
caattttggtagcagtagac

SEQ ID NO: 9 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ggttacaattttggtagcag

SEQ ID NO: 10 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gatgggggttacaattttgg

SEQ ID NO: 11 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgatgggggttacaattttg

SEQ ID NO: 12 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tactagatgatgggggttac

SEQ ID NO: 13 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttactagatgatgggggtta

SEQ ID NO: 14 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctggaatgaagcagaataca

SEQ ID NO: 15 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tctggaatgaagcagaatac

SEQ ID NO: 16 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aggcctctggaatgaagcag

SEQ ID NO: 17 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agtattctatgtgagcagct

SEQ ID NO: 18 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aagtattctatgtgagcagc

SEQ ID NO: 19 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
actggtgaaaatggaagtca

SEQ ID NO: 20 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aactggtgaaaatggaagtc

SEQ ID NO: 21 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttacagactatacagtttag

SEQ ID NO: 22 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tttacagactatacagttta

SEQ ID NO: 23 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cagactatacagtttagtag

SEQ ID NO: 24 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
acagactatacagtttagta

SEQ ID NO: 25 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tacagactatacagtttagt

SEQ ID NO: 26 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctttacagactatacagttt

SEQ ID NO: 27 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cctttacagactatacagtt

SEQ ID NO: 28 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gactgagataaatacagttc

SEQ ID NO: 29 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgactgagataaatacagtt

SEQ ID NO: 30 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
atacagtttagtagactcaa

SEQ ID NO: 31 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgcctttacagactatacag

SEQ ID NO: 32 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gcctttacagactatacagt

SEQ ID NO: 33 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctgcctttacagactataca

SEQ ID NO: 34 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tatacagtttagtagactca

SEQ ID NO: 35 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cctgcctttacagactatac

SEQ ID NO: 36 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
acttcttgagcagtaattca

SEQ ID NO: 37 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cttcttgagcagtaattcag

SEQ ID NO: 38 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttcttgagcagtaattcagc

SEQ ID NO: 39 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tacttcttgagcagtaattc

SEQ ID NO: 40 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tcttgagcagtaattcagca

SEQ ID NO: 41 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tcctgcctttacagactata

SEQ ID NO: 42 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aaggcatgtggatctatact

SEQ ID NO: 43 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cttgagcagtaattcagcat

SEQ ID NO: 44 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aggcatgtggatctatactt

SEQ ID NO: 45 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttgagcagtaattcagcatc

SEQ ID NO: 46 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctgaagttgaaggcatgtgg

SEQ ID NO: 47 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tctgaagttgaaggcatgtg

SEQ ID NO: 48 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttctgaagttgaaggcatgt

SEQ ID NO: 49 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tttaagattctgaagttgaa

SEQ ID NO: 50 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
atgtggatctatacttcttg

SEQ ID NO: 51 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgtggatctatacttcttga

SEQ ID NO: 52 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgaaggcatgtggatctata

SEQ ID NO: 53 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttgaaggcatgtggatctat

SEQ ID NO: 54 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtggatctatacttcttgag

SEQ ID NO: 55 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tctatacttcttgagcagta

SEQ ID NO: 56 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
atctatacttcttgagcagt

SEQ ID NO: 57 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tggatctatacttcttgagc

SEQ ID NO: 58 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
acagttcctgcctttacaga

SEQ ID NO: 59 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctatacttcttgagcagtaa

SEQ ID NO: 60 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ggatctatacttcttgagca

SEQ ID NO: 61 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tatacttcttgagcagtaat

SEQ ID NO: 62 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gatctatacttcttgagcag

SEQ ID NO: 63 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgagcagtaattcagcatct

SEQ ID NO: 64 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctggagtgttgctgcg

SEQ ID NO: 65 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgttcaccgtgtacca

SEQ ID NO: 66 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ggggttacaattttgg

SEQ ID NO: 67 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tatgcagactaaaaaa

SEQ ID NO: 68 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttaagattctgaagtt

SEQ ID NO: 69 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cagactaaaaaagatc

SEQ ID NO: 70 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ccacacataaagcgcc

SEQ ID NO: 71 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctcaacattcgcctct

SEQ ID NO: 72 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tttacagactatacag

SEQ ID NO: 73 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
actatacagtttagta

SEQ ID NO: 74 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctatacagtttagtag

SEQ ID NO: 75 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agttcctgcctttaca

SEQ ID NO: 76 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
taaagcgccctggagt

SEQ ID NO: 77 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gctgcgagaatttgta

SEQ ID NO: 78 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agatgaactgttatga

SEQ ID NO: 79 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttactagatgatgggg

SEQ ID NO: 80 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
taatttaagattctga

SEQ ID NO: 81 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tactagatgatggggg

SEQ ID NO: 82 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
atgcagactaaaaaag

SEQ ID NO: 83 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
acattcgcctctaatt

SEQ ID NO: 84 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tacagactatacagtt

SEQ ID NO: 85 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gactgagataaataca

SEQ ID NO: 86 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tatacagtttagtaga

SEQ ID NO: 87 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gttcctgcctttacag

SEQ ID NO: 88 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gcaggcctctggaatg

SEQ ID NO: 89 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttgggctgctctatct

SEQ ID NO: 90 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctgatctaaactggtg

SEQ ID NO: 91 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttaacacagatgaact

SEQ ID NO: 92 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctaatttaagattctg

SEQ ID NO: 93 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
actctttactagatga

SEQ ID NO: 94 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tctttactagatgatg

SEQ ID NO: 95 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tagactcaacattcgc

SEQ ID NO: 96 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctttacagactataca

SEQ ID NO: 97 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
actgagataaatacag

SEQ ID NO: 98 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttacagactatacagt

SEQ ID NO: 99 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
taatctccacatgtat

SEQ ID NO: 100 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cacataaagcgccctg

SEQ ID NO: 101 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gcattatggaaggcac

SEQ ID NO: 102 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gagattaagaattggc

SEQ ID NO: 103 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gttaacacagatgaac

SEQ ID NO: 104 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cctctaatttaagatt

SEQ ID NO: 105 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctatgtgagcagctct

SEQ ID NO: 106 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
aagattctgaagttga

SEQ ID NO: 107 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
aggcaccacagtagca

SEQ ID NO: 108 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttgcattatggaaggc

SEQ ID NO: 109 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tccactgatctaaact

SEQ ID NO: 110 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gagtgttgctgcgaga

SEQ ID NO: 111 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgttaacacagatgaa

SEQ ID NO: 112 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agattctgaagttgaa

SEQ ID NO: 113 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ataaagcgccctggag

SEQ ID NO: 114 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gggggttacaattttg

SEQ ID NO: 115 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agactatacagtttag

SEQ ID NO: 116 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgactgagataaatac

SEQ ID NO: 117 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttcagcatctctctgt

SEQ ID NO: 118 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cttaatctccacatgt

SEQ ID NO: 119 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tatgtgagcagctctt

SEQ ID NO: 120 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctagatgatgggggtt

SEQ ID NO: 121 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ataaatacagttcctg

SEQ ID NO: 122 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tttactagatgatggg

SEQ ID NO: 123 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gctgcgagaatttgta

SEQ ID NO: 124 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tttaagattctgaagt

SEQ ID NO: 125 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
aacttgtagggttaat

SEQ ID NO: 126 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttgtagggttaatatg

SEQ ID NO: 127 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cagactatacagttta

SEQ ID NO: 128 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgagataaatacagtt

SEQ ID NO: 129 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gtcttaatctccacat

SEQ ID NO: 130 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gcagtaattcagcatc

SEQ ID NO: 131 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgaaggcatgtggatc

SEQ ID NO: 132 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agtgtttgagaggagc

SEQ ID NO: 133 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agtgttgctgcgagaa

SEQ ID NO: 134 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctttactagatgatgg

SEQ ID NO: 135 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ttgctgcgagaatttg

SEQ ID NO: 136 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
atttaagattctgaag

SEQ ID NO: 137 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ccgtgtaccaactctt

SEQ ID NO: 138 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cttgtagggttaatat

SEQ ID NO: 139 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cctttacagactatac

SEQ ID NO: 140 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tacttcttgagcagta

SEQ ID NO: 141 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cagttcctgcctttac

SEQ ID NO: 142 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cagtaattcagcatct

SEQ ID NO: 143 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgcattatggaaggca

SEQ ID NO: 144 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
agactcaacattcgcc

SEQ ID NO: 145 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gtgttgctgcgagaat

SEQ ID NO: 146 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
gattctgaagttgaag

SEQ ID NO: 147 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgttgctgcgagaatt

SEQ ID NO: 148 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tggagtgttgctgcga

SEQ ID NO: 149 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
actcaacattcgcctc

SEQ ID NO: 150 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tcgcctctaatttaag

SEQ ID NO: 151 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cttcttgagcagtaattcag

SEQ ID NO: 152 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(6): 2′-methoxyethyl (MOE) modified nucleoside
  modified_base (7)..(14): 2′-deoxynucleosides
  modified_base (15)..(20): 2′-methoxyethyl (MOE) modified nucleoside
cttcttgagcagtaattcag

SEQ ID NO: 153 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(7): 2′-methoxyethyl (MOE) modified nucleoside
  modified_base (8)..(13): 2′-deoxynucleosides
  modified_base (14)..(20): 2′-methoxyethyl (MOE) modified nucleoside
cttcttgagcagtaattcag

SEQ ID NO: 154 - skipped

SEQ ID NO: 155 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(13): 2′-deoxynucleosides
  modified_base (14)..(18): 2′-methoxyethyl (MOE) modified nucleoside
ttcttgagcagtaattca

SEQ ID NO: 156 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(12): 2′-deoxynucleosides
  modified_base (13)..(16): 2′-methoxyethyl (MOE) modified nucleoside
tcttgagcagtaattc

SEQ ID NO: 157 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(11): 2′-deoxynucleosides
  modified_base (12)..(16): 2′-methoxyethyl (MOE) modified nucleoside
tcttgagcagtaattc

SEQ ID NO: 158 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(17): cMe modified nucleoside
tcttgagcagtaattca

SEQ ID NO: 159 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(13): 2′-deoxynucleosides
  modified_base (14)..(17): cMe modified nucleoside
tcttgagcagtaattca

SEQ ID NO: 160 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tcttgagcagtaattc

SEQ ID NO: 161 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(12): 2′-deoxynucleosides
  modified_base (13)..(16): cMe modified nucleoside
tcttgagcagtaattc

SEQ ID NO: 162 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(4): 2′-deoxynucleosides
  modified_base (5)..(5): 2′OMe modified nucleoside
  modified_base (6)..(13): 2′-deoxynucleosides
  modified_base (14)..(16): cMe modified nucleoside
tcttgagcagtaattc

SEQ ID NO: 163 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tactagatgatgggggttac

SEQ ID NO: 164 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(6): 2′-methoxyethyl (MOE) modified nucleoside
  modified_base (7)..(14): 2′-deoxynucleosides
  modified_base (15)..(20): 2′-methoxyethyl (MOE) modified nucleoside
tactagatgatgggggttac

SEQ ID NO: 165 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(7): 2′-methoxyethyl (MOE) modified nucleoside
  modified_base (8)..(13): 2′-deoxynucleosides
  modified_base (14)..(20): 2′-methoxyethyl (MOE) modified nucleoside
tactagatgatgggggttac

SEQ ID NO: 166 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
actagatgatgggggtta

SEQ ID NO: 167 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(13): 2′-deoxynucleosides
  modified_base (14)..(18): 2′-methoxyethyl (MOE) modified nucleoside
actagatgatgggggtta

SEQ ID NO: 168 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(12): 2′-deoxynucleosides
  modified_base (13)..(16): 2′-methoxyethyl (MOE) modified nucleoside
ctagatgatgggggtt

SEQ ID NO: 169 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(11): 2′-deoxynucleosides
  modified_base (12)..(16): 2′-methoxyethyl (MOE) modified nucleoside
ctagatgatgggggtt

SEQ ID NO: 170 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(17): cMe modified nucleoside
ctagatgatgggggtta

SEQ ID NO: 171 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(13): 2′-deoxynucleosides
  modified_base (14)..(17): cMe modified nucleoside
ctagatgatgggggtta

SEQ ID NO: 172 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
ctagatgatgggggtt

SEQ ID NO: 173 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(12): 2′-deoxynucleosides
  modified_base (13)..(16): cMe modified nucleoside
ctagatgatgggggtt

SEQ ID NO: 174 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(4): 2′-deoxynucleosides
  modified_base (5)..(5): 2′OMe modified nucleoside
  modified_base (6)..(13): 2′-deoxynucleosides
  modified_base (14)..(16): cMe modified nucleoside
ctagatgatgggggtt

SEQ ID NO: 175 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cctgcctttacagactatac

SEQ ID NO: 176 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(6): 2′-methoxyethyl (MOE) modified nucleoside
  modified_base (7)..(14): 2′-deoxynucleosides
  modified_base (15)..(20): 2′-methoxyethyl (MOE) modified nucleoside
cctgcctttacagactatac

SEQ ID NO: 177 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(7): 2′-methoxyethyl (MOE) modified nucleoside
  modified_base (8)..(13): 2′-deoxynucleosides
  modified_base (14)..(20): 2′-methoxyethyl (MOE) modified nucleoside
cctgcctttacagactatac

SEQ ID NO: 178 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
ctgcctttacagactata

SEQ ID NO: 179 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(13): 2′-deoxynucleosides
  modified_base (14)..(18): 2′-methoxyethyl (MOE) modified nucleoside
ctgcctttacagactata

SEQ ID NO: 180 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(12): 2′-deoxynucleosides
  modified_base (13)..(16): 2′-methoxyethyl (MOE) modified nucleoside
tgcctttacagactat

SEQ ID NO: 181 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(11): 2′-deoxynucleosides
  modified_base (12)..(16): 2′-methoxyethyl (MOE) modified nucleoside
tgcctttacagactat

SEQ ID NO: 182 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(17): cMe modified nucleoside
tgcctttacagactata

SEQ ID NO: 183 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(13): 2′-deoxynucleosides
  modified_base (14)..(17): cMe modified nucleoside
tgcctttacagactata

SEQ ID NO: 184 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
tgcctttacagactat

SEQ ID NO: 185 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(12): 2′-deoxynucleosides
  modified_base (13)..(16): cMe modified nucleoside
tgcctttacagactat

SEQ ID NO: 186 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(4): 2′-deoxynucleosides
  modified_base (5)..(5): 2′OMe modified nucleoside
  modified_base (6)..(13): 2′-deoxynucleosides
  modified_base (14)..(16): cMe modified nucleoside
tgcctttacagactat

SEQ ID NO: 187 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttacagactatacagtttag

SEQ ID NO: 188 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(6): 2′-methoxyethyl (MOE) modified nucleoside
  modified_base (7)..(14): 2′-deoxynucleosides
  modified_base (15)..(20): 2′-methoxyethyl (MOE) modified nucleoside
ttacagactatacagtttag

SEQ ID NO: 189 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(7): 2′-methoxyethyl (MOE) modified nucleoside
  modified_base (8)..(13): 2′-deoxynucleosides
  modified_base (14)..(20): 2′-methoxyethyl (MOE) modified nucleoside
ttacagactatacagtttag

SEQ ID NO: 190 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
tacagactatacagttta

SEQ ID NO: 191 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(13): 2′-deoxynucleosides
  modified_base (14)..(18): 2′-methoxyethyl (MOE) modified nucleoside
tacagactatacagttta

SEQ ID NO: 192 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(12): 2′-deoxynucleosides
  modified_base (13)..(16): 2′-methoxyethyl (MOE) modified nucleoside
acagactatacagttt

SEQ ID NO: 193 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(11): 2′-deoxynucleosides
  modified_base (12)..(16): 2′-methoxyethyl (MOE) modified nucleoside
acagactatacagttt

SEQ ID NO: 194 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(17): cMe modified nucleoside
acagactatacagttta

SEQ ID NO: 195 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(13): 2′-deoxynucleosides
  modified_base (14)..(17): cMe modified nucleoside
acagactatacagttta

SEQ ID NO: 196 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cagactatacagttta

SEQ ID NO: 197 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(12): 2′-deoxynucleosides
  modified_base (13)..(16): cMe modified nucleoside
cagactatacagttta

SEQ ID NO: 198 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(4): 2′-deoxynucleosides
  modified_base (5)..(5): 2′OMe modified nucleoside
  modified_base (6)..(13): 2′-deoxynucleosides
  modified_base (14)..(16): cMe modified nucleoside
cagactatacagttta

SEQ ID NO: 199 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cacacataaagcgccctgga

SEQ ID NO: 200 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(6): 2′-methoxyethyl (MOE) modified nucleoside
  modified_base (7)..(14): 2′-deoxynucleosides
  modified_base (15)..(20): 2′-methoxyethyl (MOE) modified nucleoside
cacacataaagcgccctgga

SEQ ID NO: 201 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(7): 2′-methoxyethyl (MOE) modified nucleoside
  modified_base (8)..(13): 2′-deoxynucleosides
  modified_base (14)..(20): 2′-methoxyethyl (MOE) modified nucleoside
cacacataaagcgccctgga

SEQ ID NO: 202 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
acacataaagcgccctgg

SEQ ID NO: 203 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(13): 2′-deoxynucleosides
  modified_base (14)..(18): 2′-methoxyethyl (MOE) modified nucleoside
acacataaagcgccctgg

SEQ ID NO: 204 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(12): 2′-deoxynucleosides
  modified_base (13)..(16): 2′-methoxyethyl (MOE) modified nucleoside
cacataaagcgccctg

SEQ ID NO: 205 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(11): 2′-deoxynucleosides
  modified_base (12)..(16): 2′-methoxyethyl (MOE) modified nucleoside
cacataaagcgccctg

SEQ ID NO: 206 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(17): cMe modified nucleoside
acacataaagcgccctg

SEQ ID NO: 207 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(13): 2′-deoxynucleosides
  modified_base (14)..(17): cMe modified nucleoside
acacataaagcgccctg

SEQ ID NO: 208 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cacataaagcgccctg

SEQ ID NO: 209 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(12): 2′-deoxynucleosides
  modified_base (13)..(16): cMe modified nucleoside
cacataaagcgccctg

SEQ ID NO: 210 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(4): 2′-deoxynucleosides
  modified_base (5)..(5): 2′OMe modified nucleoside
  modified_base (6)..(13): 2′-deoxynucleosides
  modified_base (14)..(16): cMe modified nucleoside
cacataaagcgccctg

SEQ ID NO: 211 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
actgagataaatacagttcc

SEQ ID NO: 212 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(6): 2′-methoxyethyl (MOE) modified nucleoside
  modified_base (7)..(14): 2′-deoxynucleosides
  modified_base (15)..(20): 2′-methoxyethyl (MOE) modified nucleoside
actgagataaatacagttcc

SEQ ID NO: 213 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(7): 2′-methoxyethyl (MOE) modified nucleoside
  modified_base (8)..(13): 2′-deoxynucleosides
  modified_base (14)..(20): 2′-methoxyethyl (MOE) modified nucleoside
actgagataaatacagttcc

SEQ ID NO: 214 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
ctgagataaatacagttc

SEQ ID NO: 215 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(13): 2′-deoxynucleosides
  modified_base (14)..(18): 2′-methoxyethyl (MOE) modified nucleoside
ctgagataaatacagttc

SEQ ID NO: 216 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(12): 2′-deoxynucleosides
  modified_base (13)..(16): 2′-methoxyethyl (MOE) modified nucleoside
tgagataaatacagtt

SEQ ID NO: 217 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(11): 2′-deoxynucleosides
  modified_base (12)..(16): 2′-methoxyethyl (MOE) modified nucleoside
tgagataaatacagtt

SEQ ID NO: 218 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(17): cMe modified nucleoside
ctgagataaatacagtt

SEQ ID NO: 219 - DNA, 17 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(13): 2′-deoxynucleosides
  modified_base (14)..(17): cMe modified nucleoside
ctgagataaatacagtt

SEQ ID NO: 220 - skipped

SEQ ID NO: 221 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): cMe modified nucleoside
  modified_base (6)..(12): 2′-deoxynucleosides
  modified_base (13)..(16): cMe modified nucleoside
tgagataaatacagtt

SEQ ID NO: 222 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(4): 2′-deoxynucleosides
  modified_base (5)..(5): 2′OMe modified nucleoside
  modified_base (6)..(13): 2′-deoxynucleosides
  modified_base (14)..(16): cMe modified nucleoside
tgagataaatacagtt

SEQ ID NO: 223 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
ttcttgagcagtaattca

SEQ ID NO: 224 - DNA, 18 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(4): 2′-methoxyethyl (MOE) modified nucleoside
  modified_base (5)..(14): 2′-deoxynucleosides
  modified_base (15)..(18): 2′-methoxyethyl (MOE) modified nucleoside
ttcttgagcagttattca

SEQ ID NO: 225 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cttgagcagtaattca

SEQ ID NO: 226 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(13): 2′-deoxynucleosides
  modified_base (14)..(16): cMe modified nucleoside
cttgagcagttattca

SEQ ID NO: 227 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cMe modified nucleoside
  modified_base (4)..(5): 2′-deoxynucleosides
  modified_base (6)..(6): 2′OMe modified nucleoside
  modified_base (7)..(13): 2′-deoxynucleosides
  modified_base (14)..(16): cMe modified nucleoside
cttgagcagtaattca

SEQ ID NO: 228 - DNA, 15 bp, Artificial Sequence
  source: /note=“Description of Artificial Sequence: Synthetic oligonucleotide”
aacacgtctatacgc

SEQ ID NO: 229 - skipped

SEQ ID NO: 230 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): 2′F-ANA modified nucleoside
  modified_base (4)..(13): 2′-deoxynucleosides
  modified_base (14)..(16): 2′F-ANA modified nucleoside
tcgcctctaatttaag

SEQ ID NO: 231 - DNA, 575 bp, Artificial Sequence
  source: /note=“Description of Artificial Sequence: Synthetic polynucleotide”
atacatgtggagattaagacccataataacaatgacaacactttcataacagttcatctgtgttaacatacaaattctcgcagcaacactccagggcgctttatgtgtggatcttttttagtctgcatattaaccctacaagttggaaatggctcctctcaaacactggagatagagcagcccaaatgtatctgctactgtggtgccttccataatgcaaaactctctgaggagctgagaatatgtctactgctaccaaaattgtaacccccatcatctagtaaagagttggtacacggtgaacatttgctgtgggaatgtattctgcttcattccagaggcctgccaattcttaatctcactataggctgaagagctgctcacatagaatacttgtagtgacttccattttcaccagtttagatcagtggacagagagatgctgaattactgctcaagaagtatagatccacatgccttcaacttcagaatcttaaattagaggcgaatgttgagtctactaaactgtatagtctgtaaaggcaggaactgtatttatctcagtcatatttaat

SEQ ID NO: 232 - DNA, 222 bp, Artificial Sequence
  source: /note=“Description of Artificial Sequence: Synthetic polynucleotide”
gttacatgtagagatggaaacttgcaataacaatggatcaaaccctcacaatgctagctgtcaccatattaggctagatgatagaaacatgtgaataactgctcaagaaaatatagaaccacatcctttgaaattcagaagcttcaactgggagggctcttgagcctgctggactgtatactctgtaaaaacagaactgtcttcgtctcactcactatttta

SEQ ID NO: 233 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctctggaatgaagcagaata

SEQ ID NO: 234 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gctctatctccagtgtttga

SEQ ID NO: 235 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gaatgaagcagaatacattc

SEQ ID NO: 236 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
atttgggctgctctatctcc

SEQ ID NO: 237 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgtgagcagctcttcagcct

SEQ ID NO: 238 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
acttgtagggttaatatgca

SEQ ID NO: 239 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gcctatagtgagattaagaa

SEQ ID NO: 240 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tagcagatacatttgggctg

SEQ ID NO: 241 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cagcctatagtgagattaag

SEQ ID NO: 242 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aaactggtgaaaatggaagt

SEQ ID NO: 243 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cattatggaaggcaccacag

SEQ ID NO: 244 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
atgcagactaaaaaagatcc

SEQ ID NO: 245 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cagaatacattcccacagca

SEQ ID NO: 246 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
caacttgtagggttaatatg

SEQ ID NO: 247 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cagagagttttgcattatgg

SEQ ID NO: 248 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gagccatttccaacttgtag

SEQ ID NO: 249 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctaaaaaagatccacacata

SEQ ID NO: 250 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtagacatattctcagctcc

SEQ ID NO: 251 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aaggcaccacagtagcagat

SEQ ID NO: 252 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agagttttgcattatggaag

SEQ ID NO: 253 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tggaaggcaccacagtagca

SEQ ID NO: 254 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tctccagtgtttgagaggag

SEQ ID NO: 255 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctgctctatctccagtgttt

SEQ ID NO: 256 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agatgaactgttatgaaagt

SEQ ID NO: 257 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgaactgttatgaaagtgtt

SEQ ID NO: 258 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtcactacaagtattctatg

SEQ ID NO: 259 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gaagcagaatacattcccac

SEQ ID NO: 260 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
atgaagcagaatacattccc

SEQ ID NO: 261 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
caagtattctatgtgagcag

SEQ ID NO: 262 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tttgggctgctctatctcca

SEQ ID NO: 263 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttgtagggttaatatgcaga

SEQ ID NO: 264 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agtagacatattctcagctc

SEQ ID NO: 265 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ggcaggcctctggaatgaag

SEQ ID NO: 266 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttctatgtgagcagctcttc

SEQ ID NO: 267 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gaggagccatttccaacttg

SEQ ID NO: 268 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tcagagagttttgcattatg

SEQ ID NO: 269 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tcctcagagagttttgcatt

SEQ ID NO: 270 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tatctccagtgtttgagagg

SEQ ID NO: 271 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ccaacttgtagggttaatat

SEQ ID NO: 272 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gatacatttgggctgctcta

SEQ ID NO: 273 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttgtcattgttattatgggt

SEQ ID NO: 274 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tatgaaagtgttgtcattgt

SEQ ID NO: 275 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tgatctaaactggtgaaaat

SEQ ID NO: 276 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
catattctcagctcctcaga

SEQ ID NO: 277 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agaggagccatttccaactt

SEQ ID NO: 278 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gaaggcaccacagtagcaga

SEQ ID NO: 279 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
catttgggctgctctatctc

SEQ ID NO: 280 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gaaaatggaagtcactacaa

SEQ ID NO: 281 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctctctgtccactgatctaa

SEQ ID NO: 282 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agagagttttgcattatgga

SEQ ID NO: 283 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tccactgatctaaactggtg

SEQ ID NO: 284 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gccatttccaacttgtaggg

SEQ ID NO: 285 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtttgagaggagccatttcc

SEQ ID NO: 286 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gagcagctcttcagcctata

SEQ ID NO: 287 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
cttgtagggttaatatgcag

SEQ ID NO: 288 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ttgagaggagccatttccaa

SEQ ID NO: 289 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
ctcctcagagagttttgcat

SEQ ID NO: 290 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
tagcagtagacatattctca

SEQ ID NO: 291 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gagttttgcattatggaagg

SEQ ID NO: 292 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
aacttgtagggttaatatgc

SEQ ID NO: 293 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtccactgatctaaactggt

SEQ ID NO: 294 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
gtagggttaatatgcagact

SEQ ID NO: 295 - DNA, 20 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(5): 2′-methoxyethyl (MOE) modified nucleoside
  modified_base (6)..(15): 2′-deoxynucleosides
  modified_base (16)..(20): 2′-methoxyethyl (MOE) modified nucleoside
agctcttcagcctatagtga

SEQ ID NO: 296 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cET modified nucleoside
  modified_base (4)..(13): 2′-deoxynucleosides
  modified_base (14)..(16): cET modified nucleoside
tcttgagcagtaattc

SEQ ID NO: 297 - DNA, 16 bp, Artificial Sequence
  source: /note=“Description of Artificial Sequence: Synthetic oligonucleotide”
  source: /note=“Description of Combined DNA/RNA Molecule: Synthetic oligonucleotide”
  modified_base (1)..(3): cET modified nucleoside
  modified_base (4)..(13): 2′-deoxynucleosides
  modified_base (14)..(16): cET modified nucleoside
cagactatacagttta

(19) United States
(12) Patent Application Publication
(10) Pub. No.: US 2023/0416734 A1
(43) Pub. Date: Dec. 28, 2023

(54) UMLILO ANTISENSE TRANSCRIPTION INHIBITORS

(71) Applicant: LEMBA BV, Nijmegen (NL)

(72) Inventors: Gabriel Virgil TURCU, Craiova (RO); Marius Andrei CIUREZ, Craiova (RO); Stephanie BERRY, Scarsdale, NY (US)

(21) Appl. No.: 18/037,234

(22) PCT Filed: Nov. 17, 2021
(86) PCT No.: PCT/IB2021/060676

§ 371 Date: May 16, 2023

Related U.S. Application Data
(60) Provisional application No. 63/235,890, filed on Aug. 23, 2021.
(60) Provisional application No. 63/115,448, filed on Nov. 18, 2020.

(51) Int. Cl. C12N 15/113

(52) U.S. Cl. CPC C12N 15/113; C12N 2310/341; C12N 2310/315; C12N 2310/321; C12N 2310/322; C12N 2310/3231; C12N 2310/3341

(57) ABSTRACT

A gapmer compound that is at least 91% complementary over its entire length to a Region A, B, C, D, E, or F of UMLILO (SEQ ID NO: 231), and that inhibits multiple acute inflammatory gene transcription regulated by the UMLILO long non-coding RNA, comprising: 5′ wing sequence having from about 3 to about 7 modified nucleosides, a central gap region sequence having from about 6 to about 15 2′-deoxynucleosides, and a 3′ wing sequence having from about 3 to about 7 modified nucleosides; wherein the gapmer nucleotides are each linked by phosphorothioate internucleoside linkages, phosphorothiolate internucleoside linkages, or combinations thereof throughout the gapmer compound; and wherein the modified nucleosides comprise 2′-methoxyethyl (2′-MOE) modification, a locked nucleic acid (LNA) modification, a 2′F-ANA modification, a 2′-O-methoxyethyl (2′OMe) modification, or combinations thereof.

### CROSS REFERENCE TO RELATED APPLICATIONS

This application claims priority to provisional application No. 63/115,448, filed on Nov. 18, 2020, and provisional application No. 63/235,890, filed on Aug. 23, 2021. The entire contents of both provisional applications are incorporated herein by reference in their entirety.

### SEQUENCE LISTING

The present application is being filed along with a Sequence Listing in electronic format. The Sequence Listing is provided as a file entitled 269607-498263_SL.txt, created on Nov. 16, 2021 which is 184,808 bytes in size. The information in the electronic format of the sequence listing is incorporated herein by reference in its entirety.

### TECHNICAL FIELD

The present disclosure provides gapmer compounds comprising a modified oligonucleotide having 12 to 29 linked nucleosides. The present disclosure also provides methods for treating a disease or condition mediated by multiple acute inflammatory gene transcription regulated by an Upstream Master LncRNA of an Inflammatory Chemokine LOcus (UMLILO) long non-coding RNA (lncRNA).

### BACKGROUND

Acute inflammatory responses are accompanied by transcription of many genes after TNF induction, including those involved in cytokine signaling (e.g., TNFAIP3; IL1A, IL-1B, IL-6); chemotaxis (e.g., CCL2; CXCL1, 2, 3, 8; CSF2; CXCR7) as well as adhesion and migration (e.g., ICAM1, 4, 5). Therefore, transcription inhibitors are needed in the art to address acute inflammation.

One potential therapeutic target area is a subset of lncRNAs, such as immune-gene priming lncRNAs or “IPLs.” One IPL, was named UMLILO because it formed chromosomal contacts with the ELR+ CXCL chemokine genes (IL-8, CXCL1, CXCL2 and CXCL3; hereafter referred to as CXCL chemokines) (Fanucchi, S., Fok, E. T., Dalla, E. et al. Immune genes are primed for robust transcription by proximal long noncoding RNAs located in nuclear compartments. Nat Genet 51, 138-150 (2019)). Therefore, there is a need for therapeutic agents to inhibit the transcription of multiple genes induced by UMLILO. The present disclosure addresses this need.

Age-related macular degeneration (AMD) is the most common cause of blindness amongst the elderly in the industrialized world. There are early stages and later stages of AMD. Late-stage AMD is divided into wet AMD and geographic atrophy (GA). Choroidal neovascularization (CNV), the hallmark of ‘wet’, ‘exudative’ or ‘neovascular’ AMD, is responsible for approximately 90% of cases of severe vision loss due to AMD. Vascular endothelial growth factor (VEGF) has been shown to play a key role in the regulation of CNV and vascular permeability. Wet AMD is currently being treated with anti-VEGF therapeutics, while for the latter there is currently no approved medical treatment.

Chimeric antigen receptor (CAR) cells are currently approved for treating various cancers. However, such CAR-T therapy have a frequent and potentially fatal side effect called severe cytokine release storm (sCRS). Tocilizumab and hormone therapy have been used to treat sCRS. But these approaches are costly and increase the risk of additional side effects such as infection. Further, monoclonal antibodies, such as tocilizumab, cannot reach damaged areas in the brain because of the brain-blood barrier. Hormone therapy can also impair CAR-T cell function and weaken therapeutic efficacy. Accordingly, there is a need for an effective therapy/method to improve safety of CAR-T cell clinical application, without affecting the efficacy of CAR-T cells.

### SUMMARY

The present disclosure provides a gapmer compound comprising 12 to 29 linked nucleosides in length comprising a 5′ wing sequence from about 3 to about 7 modified nucleosides, a central gap region sequence from about 6 to about 15 2′-deoxynucleosides, and a 3′ wing sequence from about 3 to about 7 modified nucleosides,
wherein the 5′ wing and 3′ wing modified nucleosides are selected from the group consisting of a 2′-methoxyethyl (MOE) modification, a locked nucleic acid (LNA) modification, a 2′F-ANA modification, a 2′-O-methoxyethyl (2′OMe) modification, and combinations thereof;
wherein the linked nucleosides are linked with phosphorothioate internucleoside linkages, phosphorothiolate internucleoside linkages, or combinations thereof,
and wherein the modified oligonucleotide has a nucleobase sequence that is at least 91% complementary over its entire length to Region A nucleotides 256-282, Region B nucleotides 511-540, Region C nucleotides 523-547, Region D nucleotides 441-469, Region E nucleotides 88-107, or Region F nucleotides 547-567 of UMLILO lncRNA (SEQ ID NO: 231).

Preferably, the gapmer compound has a nucleotide sequence that comprises a nucleobase sequence of any one of SEQ ID NOs: 223, 12, 21, 35-42, 55-56, 88, 100-102, 123-124, 127-128, 151-153, 155-162, 224-227, and 230. Preferably, the gapmer compound has a nucleotide sequence that consists of the nucleobase sequence of any one of SEQ ID NOs: 223, 12, 21, 35-42, 55-56, 88, 100-102, 123-124, 127-128, 151-153, 155-162, 224-227, and 230. Gapmer compounds of the present invention include a gapmer compound selected from the group consisting of: 223, 12, 21, 35-42, 55-56, 88, 100-102, 123-124, 127-128, 151-153, 155-162, 224-227, and 230. Preferably, the gapmer compound of the present disclosure includes a gapmer compound selected from the group consisting of: 223-227, 36-42, 55-56, 151-153, 155-162 and 230.

In another aspect, the invention includes a gapmer compound comprising a modified oligonucleotide consisting of 12 to 29 linked nucleosides in length, wherein the modified oligonucleotide comprises a nucleobase sequence selected from the group consisting of SEQ ID NOs: 223, 12, 21, 35-42, 55-56, 88, 100-102, 123-124, 127-128, 151-153, 155-162, 224-227, and 230, wherein the gapmer compound has a 5′ wing sequence having from about 3 to about 7 modified nucleosides, a central gap region sequence having from about 6 to about 15 2′-deoxynucleosides, and a 3′ wing sequence having from about 3 to about 7 modified nucleosides, wherein the 5′ wing and 3′ wing modified nucleosides each comprise a sugar modification selected from a 2′-methoxyethyl (2′-MOE or MOE) modification, a locked nucleic acid (LNA) modification, a 2′F-ANA modification, a 2′-O-methoxyethyl (2′OMe) modification, or combinations thereof, the linked nucleosides are linked with phosphorothioate internucleoside linkages, phosphorothiolate internucleoside linkages, or combinations thereof, and wherein the modified oligonucleotide has a nucleobase sequence that is at least 91% complementary over its entire length to a nucleotide sequence of UMLILO lncRNA wherein the UMLILO lncRNA nucleotide sequence comprises the nucleotide sequence of SEQ ID NO: 231.

The present disclosure further provides a method for treating AMD, for example, wet AMD, or cytokine storm, in a subject in need of such treatment, comprising administering to the subject, a therapeutically effective amount of a composition comprising a gapmer compound, wherein the gapmer compound comprises a modified oligonucleotide consisting of 12 to 29 linked nucleosides in length, wherein the gapmer compound has a 5′ wing sequence having from about 3 to about 7 modified nucleosides, a central gap region sequence having from about 6 to about 15 2′-deoxynucleosides, and a 3′ wing sequence having from about 3 to about 7 modified nucleosides, wherein the 5′ wing and 3′ wing modified nucleosides each comprise a sugar modification selected from a 2′-methoxyethyl (2′-MOE) modification, a locked nucleic acid (LNA) modification, a 2′F-ANA modification, a 2′-O-methoxyethyl (2′OMe) modification, or combinations thereof, the gapmer compound linked nucleosides are linked with phosphorothioate internucleoside linkages, phosphorothiolate internucleoside linkages, or combinations thereof, and wherein the modified oligonucleotide has a nucleobase sequence that is at least 91% complementary over its entire length to Region A nucleotides 256-282, Region B nucleotides 511-540, Region C nucleotides 523-547, Region D nucleotides 441-469, Region E nucleotides 88-107, or Region F nucleotides 547-567 of UMLILO lnc RNA, having a nucleotide sequence that is 100% identical to the nucleotide sequence of SEQ ID NO: 231. Preferably, the methods described are used with gapmer compounds having a modified oligonucleotide sequence as provided in any one of SEQ ID 223, 12, 21, 35-42, 55-56, 88, 100-102, 123-124, 127-128, 151-153, 155-162, 224-227, and 230. Preferably, the methods described are used with gapmer compounds having a modified oligonucleotide sequence consisting of SEQ ID NOs 223-227, 36-42, 55, 56, 151-162, or 230. Gapmer compounds which find utility in the methods for example, for the treatment of AMD or cytokine storm, described herein, include a gapmer compound selected from the group consisting of gapmer compound no. 223, 12, 21, 35-42, 55-56, 88, 100-102, 123-124, 127-128, 151-153, 155-162, 224-227, and 230.

### DETAILED DESCRIPTION

### Definitions

Unless specified otherwise, the following terms are defined as follows:
“2′-substituted nucleoside” means a nucleoside comprising a 2′-substituted sugar moiety. “2′-substituted” in reference to a sugar moiety means a sugar moiety comprising at least one 2′-substituent group other than H or OH.
“2′-deoxynucleoside” means a nucleoside comprising 2′-H furanosyl sugar moiety, as found naturally occurring in deoxyribonucleosides (DNA). A 2′-deoxynucleoside may comprise a modified nucleobase or may comprise an RNA nucleobase (e.g., uracil).
“2′-O-methoxyethyl” (also 2′-MOE, MOE, and 2′-O(CH₂)₂—OCH₃) refers to an O-methoxy-ethyl modification of the 2′ position of a furosyl ring. A 2′-O-methoxyethyl modified sugar is a modified sugar.
“2′-O-methoxyethyl nucleotide” means a nucleotide comprising a 2′-O-methoxyethyl modified sugar moiety.
“5-methyl cytosine” means a cytosine modified with a methyl group attached to a 5 position. A 5-methyl cytosine is a modified nucleobase.
“About” means plus or minus 7% of the provided value.
“Active pharmaceutical agent” means the substance or substances in a pharmaceutical composition that provide a therapeutic benefit when administered to an individual. For example, in certain embodiments gapmer compound targeted to UMLILO is an active pharmaceutical agent.
“Active target region” or “target region” means a region to which one or more active antisense compounds is targeted. “Active antisense compounds” means antisense compounds that reduce target gene transcription or resulting protein levels.
“Administering” means providing a pharmaceutical agent to an individual, and includes, but is not limited to administering by a medical professional and self-administering.
“Animal” refers to a human or non-human animal, including, but not limited to, mice, rats, rabbits, dogs, cats, pigs, and non-human primates, including, but not limited to, monkeys and chimpanzees.
“Antisense activity” means any detectable and/or measurable change attributable to the hybridization of an antisense compound to its target nucleic acid. Antisense activity is a decrease in the amount or expression of a target nucleic acid or protein encoded by such target nucleic acid compared to target nucleic acid levels or target protein levels in the absence of the antisense compound.
“Antisense compound” means an oligomeric compound capable of achieving at least one antisense activity.
“alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo; cycloaliphatic [e.g., cycloalkyl or cycloalkenyl]; heterocycloaliphatic [e.g., heterocycloalkyl or heterocycloalkenyl]; aryl; heteroaryl; alkoxy; aroyl; heteroaroyl; acyl [e.g., (aliphatic)carbonyl, (cycloaliphatic)carbonyl, or (heterocycloaliphatic)carbonyl]; nitro; cyano; amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl]; amino [e.g., aliphaticamino, cycloaliphaticamino, or heterocycloaliphaticamino]; sulfonyl [e.g., aliphatic-S(O)₂—]; sulfinyl; sulfanyl; sulfoxy; urea; thiourea; sulfamoyl; sulfamide; oxo; carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; or hydroxy. Without limitation, some examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (such as alkyl-S(O)₂-aminoalkyl); aminoalkyl; amidoalkyl; (cycloaliphatic)alkyl; or haloalkyl.
“alkylene” refers to a bifunctional alkyl group.
A “bifunctional” moiety refers to a chemical group that is attached to the main chemical structure in two places, such as a linker moiety. Bifunctional moieties can be attached to the main chemical structure at any two chemically feasible substitutable points. Unless otherwise specified, bifunctional moieties can be in either direction, e.g. the bifunctional moiety “N—O” can be attached in the —N—O— direction or the —O—N— direction.
“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.
“Chimeric antisense compound” means an antisense compound that has at least two chemically distinct regions.
“Co-administration” means administration of two or more pharmaceutical agents to an individual. The two or more pharmaceutical agents may be in a single pharmaceutical composition or may be in separate pharmaceutical compositions. Each of the two or more pharmaceutical agents may be administered through the same or different routes of administration. Co-administration encompasses parallel or sequential administration.
“Complementarity” means the capacity for pairing between nucleobases of a first nucleic acid and a second nucleic acid.
“Contiguous nucleobases” means nucleobases immediately adjacent to each other.
“Diluent” means an ingredient in a composition that lacks pharmacological activity, but is pharmaceutically necessary or desirable. For example, the diluent in an injected composition may be a liquid, e.g. saline solution.
“Dose” means a specified quantity of a pharmaceutical agent provided in a single administration, or in a specified time-period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period-of-time or continuously. Doses may be stated as the amount of pharmaceutical agent per hour, day, week, or month.
“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.
“Fully complementary” or “100% complementary” means each nucleobase of a first nucleic acid has a complementary nucleobase in a second nucleic acid. In certain embodiments, a first nucleic acid is a gapmer compound and a target nucleic acid is a second nucleic acid, for example, the nucleic acid sequence of UMLILO lncRNA.
“Complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. “Fully complementary” or “100% complementary” in reference to oligonucleotides means that oligonucleotides are complementary to another oligonucleotide or nucleic acid at each nucleoside of the oligonucleotide.
“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.
“Gapmer compound” (or gapmer as used interchangeably) means a modified oligonucleotide comprising an internal “gap” region having a plurality of DNA nucleosides positioned between external regions having one or more nucleosides, wherein the nucleosides comprising the internal region are chemically distinct from the nucleoside or nucleosides comprising the external regions. The internal region is often referred to as the “gap” and the external regions is often referred to as the “wings.” Unless otherwise indicated, the sugar moieties of the nucleosides of the gap central region of a gapmer are unmodified 2′-deoxyribosyl. Thus, the term “MOE gapmer” indicates a gapmer having a sugar motif of 2′-MOE nucleosides in both wings and a gap of 2′-deoxynucleosides. Unless otherwise indicated, a 2′-MOE gapmer may comprise one or more modified internucleoside linkages and/or modified nucleobases and such modifications do not necessarily follow the gapmer pattern of the sugar modifications. A gapmer compound includes the nucleoside sequence as indicated by a SEQ ID NO: described herein, having modified wing segments indicated by the modified sugar moieties at each modified nucleoside. As used herein, gapmer compound exemplified is identical to its respective SEQ ID NO, and may be used interchangeably. For example, gapmer compound 223 is the same as gapmer compound SEQ ID NO: 223.
“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.
“Immediately adjacent” means there are no intervening elements between the immediately adjacent elements.
“Inhibiting UMLILO” means reducing transcription of genes regulated by UMLILO, including, but not limited to, IL-8, CXCL1, CXCL2 and CXCL3.
“Individual” or “Subject” used interchangeably herein, means a human or non-human animal selected for treatment or therapy.
“Modified nucleotide base” and “modified nucleoside” refers to a deoxyribose nucleotide or ribose nucleotide that is modified to have one or more chemical moieties not found in the natural nucleic acids. Examples of modified nucleotide bases, and “modified nucleosides” are compounds of Formula Ia, Formula Ib, Formula IIa, or Formula IIb as described herein.

A “Non-bicyclic modified sugar moiety” refers to the sugar moiety of a modified nucleotide base, as described herein, wherein the chemical modifications do not involve the transformation of the sugar moiety into a bicyclic or multicyclic ring system.
“Monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.
“2′-modified sugar” means a furanosyl sugar modified at the 2′ position. Such modifications include substituents as described herein.
“Bicyclic nucleoside” (BNA) refers to a modified nucleoside comprising a bicyclic sugar moiety. Examples of bicyclic nucleosides include without limitation nucleosides comprising a bridge between the 4′ and the 2′ ribosyl ring atoms. The synthesis of bicyclic nucleosides have been disclosed in, for example, U.S. Pat. No. 7,399,845, WO/2009/006478, WO/2008/150729, US2004-0171570, U.S. Pat. No. 7,427,672, Chattopadhyaya et al., J. Org. Chem. 2009, 74, 118-134, WO 99/14226, and WO 2008/154401. The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and their preparation are also described in WO 98/39352 and WO 99/14226. Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported. One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

A “4′-2′ bicyclic nucleoside” or “4′ to 2′ bicyclic nucleoside” is a bicyclic nucleoside comprising a furanose ring comprising a bridge connecting two carbon atoms of the furanose ring connects the 2′ carbon atom and the 4′ carbon atom of the sugar ring.

A “locked nucleic acid” (LNA) is a modified nucleotide base, wherein the chemical modifications are transformation of the sugar moiety into a bicyclic or multicyclic ring system. Two specific examples of locked nucleic acid compounds are β-D-methyleneoxy nucleotides, or “constrained methyl” (cMe) nucleotides; and β-D-ethyleneoxy nucleotides, or “constrained ethyl” (cEt) nucleotides.
“Mismatch” or “non-complementary” means a nucleobase of a first oligonucleotide that is not complementary with the corresponding nucleobase of a second oligonucleotide or target nucleic acid when the first and second oligonucleotide are aligned.
“Motif” means the pattern of unmodified and/or modified sugar moieties, nucleobases, and/or internucleoside linkages, in an oligonucleotide.
“Nucleobase” means an unmodified nucleobase or a modified nucleobase. An “unmodified nucleobase” is adenine (A), thymine (T), cytosine (C), uracil (U), and guanine (G).

A “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. “Nucleobase sequence” means the order of contiguous nucleobases in a nucleic acid or oligonucleotide independent of any sugar or internucleoside linkage modification.
“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).
“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by N(H)—C(═O)— O or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.
“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.
“Pharmaceutically acceptable carriers” means physiologically and pharmaceutically acceptable carriers of compounds. Pharmaceutically acceptable carriers retain the desired biological activity of the parent compound and do not impart undesired toxicological effects thereto.
“Pharmaceutical composition” means a mixture of substances suitable for administering to an animal. For example, a pharmaceutical composition may comprise an oligomeric compound and a sterile aqueous solution. In certain embodiments, a pharmaceutical composition shows activity in free uptake assay in certain cell lines.
“Phosphorothioate linkage” means a linkage between nucleosides where the phosphodiester bond is modified by replacing one of the non-bridging oxygen atoms with a sulfur atom.
“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of a gapmer compound.
“Prodrug” means a therapeutic agent that is prepared in an inactive form that is converted to an active form within the body or cells thereof by the action of endogenous enzymes or other chemicals or conditions.
“Reducing or inhibiting the amount or activity” refers to a reduction or blockade of the transcriptional expression or activity relative to the transcriptional expression or activity in an untreated or control sample and does not necessarily indicate a total elimination of transcriptional expression or activity.
“Side effects” means physiological responses attributable to a treatment other than the desired effects. Side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.
“Single-stranded oligonucleotide” means an oligonucleotide which is not hybridized to a complementary strand.
“Sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.
“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.
“Targeting” or “targeted” means the process of design and selection of an antisense compound that will specifically hybridize to a target nucleic acid and induce a desired effect.
“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.
“Target nucleic acid” and “target RNA” mean a nucleic acid that a gapmer compound is designed to affect, such as UMLILO lncRNA.
“Target region” means a portion of a target nucleic acid to which an oligomeric compound is designed to hybridize.
“Therapeutically effective amount” means an amount of a pharmaceutical agent that provides a therapeutic benefit to an individual.
“Treat” refers to administering a pharmaceutical composition to effect an alteration or improvement of a disease, disorder, or condition.
“Weekly” means every six to eight days.
“Unmodified nucleotide” means a nucleotide composed of naturally occurring nucleobases, sugar moieties, and internucleoside linkages. An unmodified nucleotide is an RNA nucleotide (i.e. β-D-ribonucleosides) or a DNA nucleotide (i.e. β-D-deoxyribonucleoside).

Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotide Synthesis

Oligomeric compounds and phosphoramidites are made by methods well known to those skilled in the art. Oligomerization of modified and unmodified nucleosides is performed according to literature procedures for DNA like compounds (Protocols for Oligonucleotides and Analogs, Ed. Agrawal (1993), Humana Press) and/or RNA like compounds (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. Alternatively, oligomers may be purchased from various oligonucleotide synthesis companies such as, for example, Care Bay, Gen Script, or Microsynth.

Irrespective of the particular protocol used, the oligomeric compounds used in accordance with this invention may be conveniently and routinely made through the well-known technique of solid phase synthesis. Equipment for such synthesis is sold by several vendors including, for example, Applied Biosystems (Foster City, CA, USA). Any other means for such synthesis known in the art may additionally or alternatively be employed (including solution phase synthesis).

Methods of isolation and analysis of oligonucleotides are well known in the art. A 96-well plate format is particularly useful for the synthesis, isolation and analysis of oligonucleotides for small scale applications.

### EMBODIMENTS

The present disclosure provides a gapmer compound that is complementary (for example, from about 91% complementary to about 100% complementary, including 100% complementary over the entire length of the gapmer compound) to a region of UMLILO long non-coding RNA, (of equivalent length of the gapmer compound) and that inhibits multiple acute inflammatory gene transcription regulated by the UMLILO long non-coding RNA. In various embodiments of the present disclosure, a gapmer compound comprises a modified oligonucleotide of 12 to 29 linked nucleosides in length. The gapmer compound is at least 91% complementary (for example, having no more than one nucleotide mismatch (i.e. 0 or 1 mismatches) over the entire length of the gapmer compound) to a region (of equal length relative to the gapmer compound) of UMLILO (SEQ ID NO: 231), and inhibits multiple acute inflammatory gene transcription from being regulated by the UMLILO long non-coding RNA. The nucleotide mismatch in all instances, occur in one of the wing segments, but not the central gap region. The gapmer compound comprises: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides;

wherein the 5′ wing and 3′ wing modified nucleosides each comprise a sugar modification selected from the group consisting of a 2′-methoxyethyl (MOE) modification, a locked nucleic acid (LNA) modification, a 2′F-ANA modification, a 2′-O-methoxyethyl (2′OMe) modification, and combinations thereof, and wherein the gapmer compound nucleosides are each linked with phosphorothioate internucleoside linkages, phosphorothiolate internucleoside linkages, or combinations thereof over the entire length of the gapmer compound. The modified oligonucleotide of the gapmer compound has a nucleobase sequence that is at least 91% complementary over its entire length to Region A of UMLILO lnc RNA, nucleotides 256-282, Region B of UMLILO lnc RNA, nucleotides 511-540, Region C of UMLILO lnc RNA, nucleotides 523-547, Region D of UMLILO lnc RNA, nucleotides 441-469, Region E of UMLILO lnc RNA, nucleotides 88-107, or Region F, nucleotides 547-567 of UMLILO long non-coding (lnc) RNA of SEQ ID NO: 231. The gapmer compounds have a nucleotide sequence over its entire length that is at least 91% complementary to the nucleotide sequence of SEQ ID NO: 231, for example, 91%, 92%, 93%, 94%, 95%, 96%, 97%, 98%, 99%, or 100% complementary to one of the Regions A-F described herein.

Preferably, the gapmer compound has a modified nucleoside sequence selected from the group consisting of SEQ ID NOs: 223, 12, 21, 35-42, 55, 56, 88, 100-102, 123, 124, 127, 128, 151-153, 155-162, 224-227 and 230.

The present disclosure provides a gapmer compound that is complementary to Region D of UMLILO (SEQ ID NO: 231 bases 441 to 469), and that inhibits multiple acute inflammatory gene transcription regulated by the UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleotides are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. Preferably, the nucleoside sequence of the gapmer compound that bind to Region D and inhibits multiple acute inflammatory gene transcription regulated by the UMLILO lncRNA is selected from the group consisting of SEQ ID NOs: 223-227, 36-42, 55, 56, 151-153, 155-162, and 230. Gapmer compounds of the present disclosure that bind to Region D, and useful in the methods described herein, include gapmer compounds 223-227, 36-42, 55, 56, 151-153, 155-162, and 230.

The present disclosure provides a gapmer compound that is complementary to Region A of UMLILO (SEQ ID NO: 231 bases 256 to 282), and that inhibits multiple acute inflammatory gene transcription regulated by the UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleosides are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. Preferably, the nucleoside sequence of the gapmer compound that bind to Region A and inhibits multiple acute inflammatory gene transcription regulated by the UMLILO lncRNA is SEQ ID NO: 12. A gapmer compound of the present disclosure that binds to Region A, and useful in the methods described herein, include gapmer compound 12.

The present disclosure provides a gapmer compound that is complementary to Region B of UMLILO (SEQ ID NO: 231 bases 511 to 540), and that inhibits multiple acute inflammatory gene transcription regulated by the UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleotises are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. Preferably, the nucleoside sequence of the gapmer compound that binds to Region B and inhibits multiple acute inflammatory gene transcription regulated by the UMLILO lncRNA is SEQ ID NO: 21. A gapmer compound of the present disclosure that binds to Region B, and useful in the methods described herein, include gapmer compound 21.

The present disclosure provides a gapmer compound that is complementary to Region C of UMLILO (SEQ ID NO: 231 bases 532 to 547), and that inhibits multiple acute inflammatory gene transcription from UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleosides are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. Preferably, the nucleoside sequence of the gapmer compound that binds to Region C and inhibits multiple acute inflammatory gene transcription regulated by the UMLILO lncRNA is SEQ ID NO: 35. A gapmer compound of the present disclosure that binds to Region C, and useful in the methods described herein, include gapmer compound 35.

The present disclosure provides a gapmer compound that is complementary to Region E of UMLILO (SEQ ID NO: 231, bases 88 to 107), and that inhibits multiple acute inflammatory gene transcription from UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleotises are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. Preferably, the nucleoside sequence of the gapmer compound that binds to Region E and inhibits multiple acute inflammatory gene transcription regulated by the UMLILO lncRNA is SEQ ID NO: 100. A gapmer compound of the present disclosure that binds to Region E, and useful in the methods described herein, include gapmer compound 100.

The present disclosure provides a gapmer compound that is complementary to Region F of UMLILO (SEQ ID NO: 231 bases 547 to 567), and that inhibits multiple acute inflammatory gene transcription regulated by the UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleosides are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. Preferably, the nucleoside sequence of the gapmer compound that binds to Region F and inhibits multiple acute inflammatory gene transcription regulated by the UMLILO lncRNA is SEQ ID NO: 128. A gapmer compound of the present disclosure that binds to Region F, and useful in the methods described herein, include gapmer compound 128.

The present disclosure provides a gapmer compound having at least 91% sequence complementarity over its entire length to target UNMILO SEQ ID NO: 231, comprising: (a) a 5′ wing sequence having from about 3 to about 7 modified nucleosides, each modified nucleoside having a modified sugar selected from the group consisting of 2′-MOE, a tetrahydropyran ring replacing a furanose ring, a bicyclic sugar with or without a 4′-CH(CH₃)—O-2′ bridge, a constrained ethyl nucleoside (cEt), a nucleoside mimetic, and combinations thereof, (b) a central gap region sequence having from about 8 to about 15 2′ deoxynucleosides; and (c) a 3′ wing sequence having from at least 3 to about 6 modified nucleosides, each nucleoside having a modified sugar selected from the group consisting of 2′-MOE, a tetrahydropyran ring replacing a furanose ring, a bicyclic sugar with or without a 4′-CH(CH₃)—O-2′ bridge, a constrained ethyl nucleoside (cEt), a nucleoside mimetic, and combinations thereof, wherein the gapmer nucleosides are each linked by phosphorothioate internucleotide bonds throughout the gapmer. Preferably the gapmer compound central gap region is a ten-nucleotide sequence from nucleotide 5 to nucleotide 15 from a sequence selected from the group consisting of SEQ ID NOs 223, 36-42, 55, 56, 151-153, 155-162, 224-227, 230 or an 8 or 9 mer fragment thereof. Preferably, the 5′ and 3′ wing modified nucleosides are a 2′-substituted nucleoside. More preferably, the 5′ and 3′ wing modified modified nucleosides are a 2′-MOE nucleoside.

In some exemplary embodiments, the present disclosure provides a gapmer compound, or a pharmaceutically acceptable carrier thereof, comprising a modified oligonucleotide consisting of 12 to 24 linked nucleosides in length, wherein the gapmer compound has a 5′ wing sequence having from about 3 to about 7 modified nucleosides, a central gap region sequence having from about 6 to about 10 2′-deoxynucleosides, and a 3′ wing sequence having from about 3 to about 7 modified nucleosides,

wherein the 5′ wing and 3′ wing modified nucleosides each comprise a sugar modification selected from a 2′-methoxyethyl (MOE) modification, a locked nucleic acid (LNA) modification, a 2′F-ANA modification, a 2′-O-methoxyethyl (2′OMe) modification, or combinations thereof,
the linked nucleosides are linked with phosphorothioate internucleoside linkages, phosphorothiolate internucleoside linkages, or combinations thereof; and wherein the gapmer compound has a nucleobase sequence that is at least 91% complementary over its entire length to Region A nucleotides 256-282, Region B nucleotides 511-540, Region C nucleotides 523-547, Region D nucleotides 441-469, Region E nucleotides 88-107, or Region F nucleotides 547-567 of UMLILO lncRNA, wherein the UMLILO lncRNA has a nucleotide sequence of SEQ ID NO: 231.

In one embodiment, the gapmer compound has zero to one mismatch over its entire length to Region D nucleotides 441-469 of SEQ ID NO: 231.

In a further embodiment, the gapmer compound is at least 100% complementary over its entire length to Region D nucleotides 441-469 of SEQ ID NO: 231.

In another embodiment, the gapmer compound has zero to one mismatch over its entire length to Region A nucleotides 256-282 of SEQ ID NO: 231.

In a further embodiment, the gapmer compound is at least 100% complementary over its entire length to Region A nucleotides 256-282 of SEQ ID NO: 231.

In another embodiment, the gapmer compound has zero to one mismatch over its entire length to Region B nucleotides 511-540 of SEQ ID NO: 231.

In a further embodiment, the gapmer compound is at least 100% complementary over its entire length to Region B nucleotides 511-540 of SEQ ID NO: 231.

In another embodiment, the gapmer compound has zero to one mismatch over its entire length to Region C nucleotides 523-547 of SEQ ID NO: 231.

In a further embodiment, the gapmer compound is at least 100% complementary over its entire length to Region C nucleotides 523-547 of SEQ ID NO: 231.

In another embodiment, the gapmer compound has zero to one mismatch over its entire length to Region E nucleotides 88-107 of SEQ ID NO: 231.

In a further embodiment, the gapmer compound is at least 100% complementary over its entire length to Region E nucleotides 88-107 of SEQ ID NO: 231.

In another embodiment, the gapmer compound has zero to one mismatch over its entire length to Region F nucleotides 547-567 of SEQ ID NO: 231.

In a further embodiment, the gapmer compound is at least 100% complementary over its entire length to Region F nucleotides 547-567 of SEQ ID NO: 231.

In one embodiment, the gapmer compound sequence comprises a modified nucleoside sequence of any one of SEQ ID NOs 223-227, 36-42, 55, 56, 151-153, 155-162, or 230.

In one embodiment, the gapmer compound is 18 linked nucleosides in length and has a nucleobase sequence consisting of the nucleobase sequence of SEQ ID NO: 223, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

In another embodiment, the gapmer compound is 18 linked nucleosides in length and has the nucleobase sequence consisting of the nucleobase sequence of SEQ ID NO: 224, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

In one embodiment, the gapmer compound is 16 linked nucleosides in length and has the nucleobase sequence consisting of the nucleobase sequence of SEQ ID NO: 225, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the 5′ wing segment consists of three locked nucleic acid (LNA) modified nucleosides; wherein the 3′ wing segment consists of three locked nucleic acid (LNA) modified nucleosides modified nucleosides; wherein each internucleoside linkage is a phosphorothioate linkage; and wherein each cytosine is a 5-methylcytosine.

In another embodiment, the gapmer compound is 16 linked nucleosides in length and has the nucleobase sequence consisting of the nucleobase sequence of SEQ ID NO: 226, wherein the modified oligonucleotide has a gap segment consisting of ten linked deoxynucleosides; a 5′ wing segment consisting of three locked nucleosides; and a 3′ wing segment consisting of three locked nucleosides; wherein the gap segment is positioned between the 5′ wing segment and the 3′ wing segment in the 5′ to 3′ direction; wherein the 5′ wing segment consists of three locked nucleic acid (LNA) modified nucleosides; wherein the 3′ wing segment consists of three locked nucleic acid (LNA) modified nucleosides modified nucleosides; wherein each internucleoside linkage is a phosphorothioate linkage; and wherein each cytosine is a 5-methylcytosine.

In another embodiment, the gapmer compound is 16 linked nucleosides in length and has the nucleobase sequence consisting of the nucleobase sequence of SEQ ID NO: 227, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the gap segment consists of nine deoxynucleosides and one 2′-O-methoxyethyl (2′-MOE) modified nucleoside at position 3 of the ten nucleosides starting from the 5′ position of the gap segment, the 5′ wing segment consists of three locked nucleic acid (LNA) modified nucleosides (cMe); wherein the 3′ wing segment consists of three locked nucleic acid (LNA) modified nucleosides (cMe); wherein each internucleoside linkage is a phosphorothioate linkage; and wherein each cytosine is a 5-methylcytosine.

In another embodiment, the gapmer compound is 16 linked nucleosides in length and has the nucleobase sequence consisting of the nucleobase sequence of SEQ ID NO: 150, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the gap segment consists of ten deoxynucleosides, the 5′ wing segment consists of three locked nucleic acid (LNA) modified nucleosides (cMe); wherein the 3′ wing segment consists of three locked nucleic acid (LNA) modified nucleosides (cMe); wherein each internucleoside linkage is a phosphorothioate linkage; and wherein each cytosine is a 5-methylcytosine.

In some embodiments, the invention includes a gapmer compound comprising a modified oligonucleotide consisting of 12 to 29 linked nucleosides in length, wherein the modified oligonucleotide comprises a nucleoside sequence selected from the group consisting of SEQ ID NOs: 223-227, 12, 21, 35-42, 55, 56, 88, 100-102, 123-124, 127-128, 151-153, 155-162, and 230, wherein the gapmer compound has a 5′ wing sequence having from about 3 to about 7 modified nucleosides, a central gap region sequence having from about 6 to about 10 2′-deoxynucleosides, and a 3′ wing sequence having from about 3 to about 7 modified nucleosides, wherein the 5′ wing and 3′ wing modified nucleosides each comprise a sugar modification selected from a 2′-methoxyethyl (MOE) modification, a locked nucleic acid (LNA) modification, a 2′F-ANA modification, a 2′-O-methoxyethyl (2′OMe) modification, or combinations thereof,

the linked nucleosides are linked with phosphorothioate internucleoside linkages, phosphorothiolate internucleoside linkages, or combinations thereof; and wherein the modified oligonucleotide has a nucleobase sequence that is at least 91% complementary (i.e. the gapmer compound has 0 or at most, 1 mismatch, for example, at least 95%, 96%, 97%, 98%, 99%, or at least 100% complementary with SEQ ID NO: 231) over its entire length, to a nucleotide sequence of Upstream Master LncRNA Of The Inflammatory Chemokine Locus (UMLILO) long non-coding RNA, wherein the UMLILO long non-coding RNA nucleotide sequence has a nucleotide sequence of SEQ ID NO: 231. The mismatch only occurs in one of the wing segments, but not in the central gap region.

In one embodiment, the gapmer compound of the present disclosure includes any one of gapmer compound no. 223-227, 12, 21, 35-42, 55, 56, 88, 100-102, 123-124, 127-128, 151-153, 155-162, and 230 as provided in Table 1.

In one embodiment, the gapmer compound is 18 linked nucleosides in length and has a nucleobase sequence consisting of the nucleobase sequence of SEQ ID NO: 223, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

In another embodiment, the gapmer compound is 18 linked nucleosides in length and has the nucleobase sequence consisting of the nucleobase sequence of SEQ ID NO: 224, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

In one embodiment, the modified oligonucleotide is 16 linked nucleosides in length and has the nucleobase sequence consisting of the nucleobase sequence of SEQ ID NO: 230, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the 5′ wing segment consists of three 2′F-ANA modified nucleosides; wherein the 3′ wing segment consists of three 2′F-ANA modified nucleosides; wherein each internucleoside linkage is a phosphorothioate linkage; and wherein each cytosine is a 5-methylcytosine.

In one embodiment, the locked nucleic acid (LNA) modification is selected from a constrained ethyl (cEt) modification and a constrained methyl (cMe) modification.

In some embodiments, the gapmer compounds described herein, have a nucleobase sequence, wherein the cytosine is a 5-methylcytosine.

The present disclosure provides a method for treating AMD or cytokine storm comprising administering a therapeutically effective amount of a gapmer compound that is at least 91% complementary over its entire length of the gapmer compound modified oligonucleotide to a region (of equal length relative to the length of the gapmer compound) of UMLILO (SEQ ID NO: 231), and that inhibits multiple acute inflammatory gene transcription regulated by the UMLILO long non-coding RNA, the gapmer compound comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleosides are each linked by phosphorothioate internucleoside linkages, phosphorothiolate internucleoside linkages, or combinations thereof throughout the gapmer; and wherein the modified nucleoside modifications are selected from the group consisting of 2′-methoxyethyl (MOE) nucleotides, locked nucleic acid nucleotides (LNA), and combinations thereof. The modified oligonucleotide of the gapmer compound has a nucleobase sequence that is at least 91% complementary over its entire length to Region A of UMLILO lnc RNA, nucleotides 256-282, Region B of UMLILO Inc RNA, nucleotides 511-540, Region C of UMLILO lnc RNA, nucleotides 523-547, Region D of UMLILO lnc RNA, nucleotides 441-469, Region E of UMLILO lnc RNA, nucleotides 88-107, or Region F, nucleotides 547-567 of UMLILO long non-coding (lnc) RNA of SEQ ID NO: 231. The gapmer compounds have a nucleotide sequence over its entire length that is at least 91% complementary to the nucleotide sequence of SEQ ID NO: 231, for example, 91%, 92%, 93%, 94%, 95%, 96%, 97%, 98%, 99%, or 100% complementary to one of the Regions A-F of UMLILO (SEQ ID NO: 231) described herein. Most preferably, the gapmer compound is at least 91% complementary (over the entire length of the gapmer compound) to a part (of equivalent length relative to the length of the gapmer compound) of Region D bases 441-469 of SEQ ID NO: 231. Gapmer compounds which find utility in the methods for example, for the treatment of AMD or cytokine storm, described herein, include a gapmer compound selected from the group consisting of gapmer compound no. 223, 12, 21, 35-42, 55-56, 88, 100-102, 123-124, 127-128, 151-153, 155-162, 224-227, and 230.

The present disclosure provides a method for treating age-related macular degeneration, for example, wet-AMD, comprising administering a therapeutically effective amount of a gapmer compound that is at least 91% complementary over its entire length of the gapmer compound modified oligonucleotide to a region of UMLILO (SEQ ID NO: 231), and that inhibits multiple acute inflammatory gene transcription from UMLILO long non-coding RNA, comprising: (a) a 5′ wing sequence having from about 3 to about 7 modified nucleosides, (b) a central gap region sequence having from about 6 to about 15 2′-deoxynucleosides, and (c) a 3′ wing sequence having from about 3 to about 7 modified nucleosides; wherein the gapmer nucleosides are each linked by phosphorothioate internucleotide bonds throughout the gapmer; and wherein the modified nucleoside modifications are selected from the group consisting of 2′-methoxyethyl (MOE) nucleosides, locked nucleic acid nucleosides (LNA), and combinations thereof. Preferably, the gapmer compound has a nucleoside sequence selected from the group consisting of SEQ ID NOs: 223, 12, 21, 35-42, 55, 56, 88, 100-102, 123, 124, 127, 128, 151-153, 155-162, 224-227 and 230. The regions of the UMLILO sequence are selected from the group consisting of Region A bases 256-282, Region B bases 511-540, Region C bases 523-547, Region D bases 441-469, Region E bases 88-107, and Region F bases 547-567. Most preferably, the gapmer is complementary to a part of Region D bases 441-469. Gapmer compounds which find utility in the methods for the treatment of AMD, for example, wet-AMD, described herein, include administration od a therapeutically effective amount of a gapmer compound selected from the group consisting of gapmer compound no. 223, 12, 21, 35-42, 55-56, 88, 100-102, 123-124, 127-128, 151-153, 155-162, 224-227, and 230.

In one embodiment, the gapmer compound useful in the treatment of AMD or cytokine storm includes administering to a subject with AMD or cytokine storm, a therapeutically effective amount of a composition comprising a gapmer compound having a modified oligonucleotide sequence comprising any one of SEQ ID NOs 223, 12, 21, 35-42, 55-56, 88, 100-102, 123-124, 127-128, 151-153, 155-162, 224-227, and 230, and a pharmaceutically acceptable excipient. Preferably, the gapmer compound useful in the treatment of AMD or cytokine storm, includes administering a therapeutically effective amount of a composition comprising a gapmer compound selected from the group consisting of gapmer compound 223, 12, 21, 35-42, 55-56, 88, 100-102, 123-124, 127-128, 151-153, 155-162, 224-227, and 230, more preferably, a therapeutically effective amount of a gapmer compound selected from the group consisting of gapmer compound 223-227, 36-42, 55-56, 151, 153, 155-162, and 230.

UMLILO Target

The UMLILO RNA sequence (SEQ ID NO: 231) is 575 bases in length and has the following sequence:

5′ATACATGTGGAGATTAAGACCCATAATAACAATGACAACACTTTCAT
AACAGTTCATCTGTGTTAACATACAAATTCTCGCAGCAACACTCCAGGG
CGCTTTATGTGTGGATCTTTTTTAGTCTGCATATTAACCCTACAAGTTG
GAAATGGCTCCTCTCAAACACTGGAGATAGAGCAGCCCAAATGTATCTG
CTACTGTGGTGCCTTCCATAATGCAAAACTCTCTGAGGAGCTGAGAATA
TGTCTACTGCTACCAAAATTGTAACCCCCATCATCTAGTAAAGAGTTGG
TACACGGTGAACATTTGCTGTGGGAATGTATTCTGCTTCATTCCAGAGG
CCTGCCAATTCTTAATCTCACTATAGGCTGAAGAGCTGCTCACATAGAA
TACTTGTAGTGACTTCCATTTTCACCAGTTTAGATCAGTGGACAGAGAG
ATGCTGAATTACTGCTCAAGAAGTATAGATCCACATGCCTTCAACTTCA
GAATCTTAAATTAGAGGCGAATGTTGAGTCTACTAAACTGTATAGTCTG
TAAAGGCAGGAACTGTATTTATCTCAGTCATATTTAAT 3′.

Length

The disclosed gapmer compounds are modified oligonucleotides having 12-29 linked nucleotides, having a gap segment of 6-15 linked deoxynucleotides between two wing segments that each wing segment each independently have 3-7 linked modified nucleosides. Preferably, the modification of the modified nucleoside in the wing segment is selected from MOE, 2′-OMe, 2′F-ANA, cMe, and cEt.

Preferably, the gapmer compound comprises:
(i) a gap segment consisting of linked deoxynucleosides;
(ii) a 5′ wing segment consisting of linked modified nucleosides;
(iii) a 3′ wing segment consisting of linked modified nucleosides, wherein the gap segment is positioned between the 5′ wing segment and the 3′ wing segment and wherein each modified nucleoside of each wing segment comprises a modified sugar: and
(iv) optionally, wherein cytidine residues are 5-methylcytidines.

Preferably, the gapmer compound comprises:
(i) a gap segment consisting of ten linked deoxynucleosides;
(ii) a 5′ wing segment consisting of five linked nucleosides;
(iii) a 3′ wing segment consisting of five linked nucleosides, wherein the gap segment is positioned immediately adjacent to and between the 5′ wing segment and the 3′ wing segment, wherein each modified nucleoside of each wing segment comprises a 2′-O-methoxyethyl sugar and/or a locked nucleic acid modified nucleoside; and wherein each internucleoside linkage is a phosphorothioate linkage: and
(iv) optionally, wherein cytidine residues are 5-methylcytidines.

Preferably, the gapmer compound comprises:
(i) a gap segment consisting of ten linked deoxynucleosides;
(ii) a 5′ wing segment consisting of four linked nucleosides;
(iii) a 3′ wing segment consisting of four linked nucleosides, wherein the gap segment is positioned immediately adjacent to and between the 5′ wing segment and the 3′ wing segment, wherein each modified nucleoside of each wing segment comprises a 2′-O-methoxyethyl (2′-MOE) sugar or a locked nucleic acid modified nucleoside (LNA); and wherein each internucleoside linkage is a phosphorothioate linkage: and
(iv) optionally, wherein cytidine residues are 5-methylcytidines.

Preferably, the gapmer compound comprises:
(i) a gap segment consisting of eight linked deoxynucleosides;
(ii) a 5′ wing segment consisting of six linked nucleosides;
(iii) a 3′ wing segment consisting of five linked nucleosides, wherein the gap segment is positioned immediately adjacent to and between the 5′ wing segment and the 3′ wing segment, wherein each modified nucleoside of each wing segment comprises a 2′-O-methoxyethyl sugar or a locked nucleic acid modified nucleoside; and wherein each internucleoside linkage is a phosphorothioate linkage: and
(iv) optionally, wherein cytidine residues are 5-methylcytidines.

Preferably, the gapmer compound comprises:
(i) a gap segment consisting of eight linked deoxynucleosides;
(ii) a 5′ wing segment consisting of five linked nucleosides;
(iii) a 3′ wing segment consisting of five linked nucleosides, wherein the gap segment is positioned immediately adjacent to and between the 5′ wing segment and the 3′ wing segment, wherein each modified nucleoside of each wing segment comprises a 2′-O-methoxyethyl sugar and/or a locked nucleic acid modified nucleoside; and wherein each internucleoside linkage is a phosphorothioate linkage: and
(iv) optionally, wherein cytidine residues are 5-methylcytidines.

Preferably, the gapmer compound comprises:
(i) a gap segment consisting of ten linked deoxynucleosides;
(ii) a 5′ wing segment consisting of five linked nucleosides;
(iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage; and wherein the nucleobase sequence comprises at least 8 contiguous nucleobases of the nucleobase sequence recited in SEQ ID NOs: 1-297.

Preferably, the gapmer compound comprises:
(i) a gap segment consisting of eight to ten (8, or 9, or 10) linked deoxynucleosides;
(ii) a 5′ wing segment consisting of three to five (3, or 4, or 5) linked nucleosides;
(iii) a 3′ wing segment consisting of three to five (3, or 4, or 5) linked nucleosides, wherein the gap segment is positioned immediately adjacent to and between the 5′ wing segment and the 3′ wing segment, wherein each modified nucleoside of each wing segment comprises a 2′-O-methoxyethyl sugar and/or a locked nucleic acid modified nucleoside; and wherein each internucleoside linkage is a phosphorothioate linkage: and
(iv) optionally, wherein cytidine residues are 5-methylcytidines, and wherein the nucleobase sequence of the gapmer compound is recited in any one of SEQ ID NOs: 1-297.

Antisense Compound Motifs

In a gapmer an internal region having a plurality of nucleotides or linked nucleosides is positioned between external regions having a plurality of nucleotides or linked nucleosides that are chemically distinct from the nucleotides or linked nucleosides of the internal region. In the case of an antisense oligonucleotide having a gapmer motif, the gap segment generally serves as the substrate for endonuclease cleavage, while the wing segments comprise modified nucleosides. The regions of a gapmer (5′ wing, gap sequence, and 3′ wing) are differentiated by the types of sugar moieties comprising each distinct region. The types of sugar moieties that are used to differentiate the regions of a gapmer may include β-D-ribonucleosides, β-D-deoxyribonucleosides, 2′-modified nucleosides (such 2′-modified nucleosides may include 2′-MOE, and 2′-OCH₃, among others), and bicyclic sugar modified nucleosides (such bicyclic sugar modified nucleosides may include those having a 4′-(CH₂)ₙ—O-2′ bridge, where n=1 or n=2). Preferably, each distinct region comprises uniform sugar moieties. The wing-gap-wing motif is frequently described as “X—Y—Z”, where “X” represents the length of the 5′ wing region, “Y” represents the length of the gap region, and “Z” represents the length of the 3′ wing region. In general, a gapmer described as “X—Y—Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Each of the gapmer compounds 36-42, 55, 56, 151-162, 223-227, 230 described have a gapmer motif Often, X and Z are the same chemistry of modified sugars as part of the nucleoside, or they are different. Preferably, Y is between 8 and 15 nucleotides. X or Z can be any of 3, 4, 5, 6, 7, 8, 9, or 10 nucleotides. Thus, gapmer compounds include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6 or 5-8-5.

In a preferred embodiment, a gapmer compound has a gap segment of ten 2′-deoxyribonucleotides positioned immediately adjacent to and between wing segments of four or five chemically modified nucleosides. In certain embodiments, the chemical modification in the wings comprises a 2′-sugar modification. In another embodiment, the chemical modification comprises a 2′-MOE or LNA sugar modification. Preferably, a gapmer compound has a gap segment of eight 2′-deoxyribonucleotides positioned immediately adjacent to and between wing segments of four or five chemically modified nucleosides, and wherein the chemical modification comprises a 2′-MOE or LNA sugar modification.

In another embodiment, a gapmer compound has a gap segment of eight 2′-deoxyribonucleotides positioned immediately adjacent to and between wing segments of four to six chemically modified nucleosides. The chemical modification comprises a 2′-MOE or LNA sugar modification.

Hybridization

Hybridization occurs between a gapmer compound and a target UMLILO nucleic acid [SEQ ID NO: 231]. The most common mechanism of hybridization involves hydrogen bonding (e.g., Watson-Crick, Hoogsteen or reversed Hoogsteen hydrogen bonding) between complementary nucleobases of the nucleic acid molecules. Hybridization can occur under varying conditions. Stringent conditions are sequence-dependent and are determined by the nature and composition of the nucleic acid molecules to be hybridized.

Modified Sugar Moieties

Gapmer compounds contain one or more nucleosides wherein the sugar group has been modified. Such sugar modified nucleosides may impart enhanced nuclease stability, increased binding affinity, or some other beneficial biological property to the antisense compounds. Nucleosides comprise chemically modified ribofuranose ring moieties. Examples of chemically modified ribofuranose rings include, without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (WO2008/101157 for other disclosed 5′,2′-bis substituted nucleosides) or replacement of the ribosyl ring oxygen atom with S with further substitution at the 2′-position (U.S. Patent Application 2005/0130923) or alternatively 5′-substitution of a BNA (WO2007/134181 wherein LNA is substituted with for example a 5′-methyl or a 5′-vinyl group).

Modified Nucleotide Bases

In one aspect, the present invention includes gapmer compounds that have modified nucleotide bases of Formula Ia Formula Ib, Formula IIa, or Formula IIb:

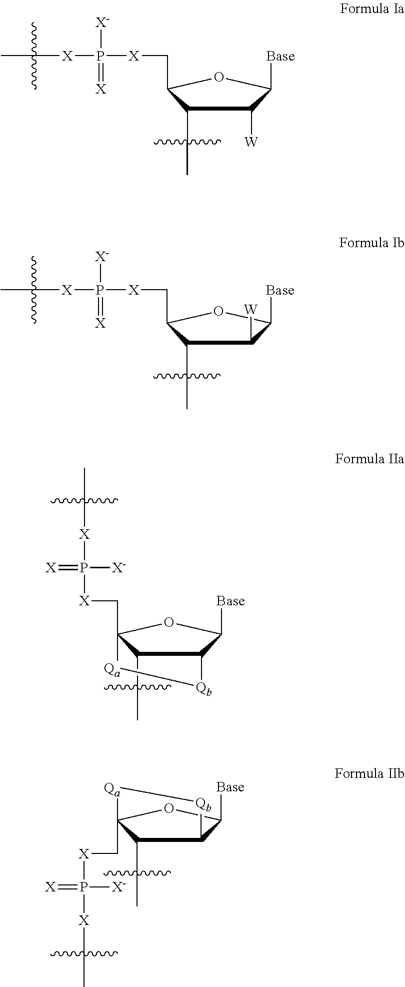

wherein
each X is independently O or S, wherein 0, 1, or 2 instances of X is S;
each W is independently H, OH, halo, or —O—C₁₋₆ alkyl, wherein the alkyl is optionally substituted with up to three instances of C₁₋₄ alkyl, C₁₋₄ alkoxy, halo, amino, CN, NO₂, or OH;
   each Qₐ is independently a bifunctional C₁₋₆ alkylene, optionally substituted with up to two instances of C₁₋₄ alkyl, C₁₋₄ alkoxy, halo, or OH; and
   each Q_{b} is independently a bond or a bifunctional moiety selected from —O—, —S—, —N—O—, —N(R)—, —C(O)—, —C(O)O—, and —C(O)N(R)—, wherein R is an unsubstituted C₁₋₄ alkyl.

In one embodiment, each X is O. In another embodiment, one instance of X is S.

In one embodiment, the gapmer compound comprises one or more nucleotides of Formula Ia or Formula Ib, wherein W is halo. In a further embodiment, W is fluoro. In another further embodiment, the gapmer compound comprises one or more nucleotides of Formula Ia. In another further embodiment, the gapmer compound comprises one or more nucleotides of Formula Ib.

In one embodiment, the gapmer compound comprises one or more nucleotides of Formula Ia or Formula Ib, wherein W is —O—C₁₋₆ alkyl, wherein the alkyl is optionally substituted with up to three instances of C₁₋₄ alkyl, C₁₋₄ alkoxy, halo, amino, or OH. In a further embodiment, W is —O—C₁₋₆ alkyl, wherein the alkyl is optionally substituted with C₁₋₄ alkoxy. In a further embodiment, W is an unsubstituted —O—C₁₋₆ alkyl. In another further embodiment, W is —O—C₁₋₆ alkyl, wherein the alkyl is substituted with C₁₋₄ alkoxy. In a further embodiment, W is selected from methoxy and —O—CH₂CH₂—OCH₃. In one embodiment, the gapmer compound comprises one or more nucleotides of Formula Ia. In another embodiment, the gapmer compound comprises one or more nucleotides of Formula Ib.

In one embodiment, the gapmer compound comprises one or more β-D nucleotides of Formula IIa or α-L nucleotides of Formula IIb, wherein Qₐ is an unsubstituted bifunctional C₁₋₆ alkylene, and Q_{b} is a bond or a bifunctional moiety selected from —O—, —S—, —N—O—, and —N(R)—. In a further embodiment, Qₐ is selected from —CH₂—, —CH₂—CH₂—, —CH(CH₃)—, —CH₂—CH₂(CH₃)—, and Q_{b} is a bond or a bifunctional moiety selected from —O—, —S—, —N(R)—O—, and —N(R)—, wherein R is H or C₁₋₆ alkyl.

In one embodiment of Formula IIa or Formula IIb, Qₐ is —CH₂— and Q_{b} is —O—. In another embodiment of Formula IIa or Formula IIb, Qₐ is —CH₂—CH₂— and Q_{b} is —O—. In another embodiment of Formula IIa or Formula IIb, Qₐ is —CH₂— and Q_{b} is —N(R)—O—, wherein R is H or C₁₋₆ alkyl. In another embodiment of Formula IIa or Formula IIb, Qₐ is —CH(CH₃)— and Q_{b} is —O—. In another embodiment of Formula IIa or Formula IIb, Qₐ is —CH₂— and Q_{b} is —S—. In another embodiment of Formula IIa or Formula IIb, Qₐ is —CH₂— and Q_{b} is —N(R)—, wherein R is H or C₁₋₆ alkyl. In another embodiment of Formula IIa or Formula IIb, Qₐ is —CH₂—CH(CH₃)— and Q_{b} is a bond.

In some embodiments, the gapmer compound comprises one or more nucleotides selected from the following nucleotides:

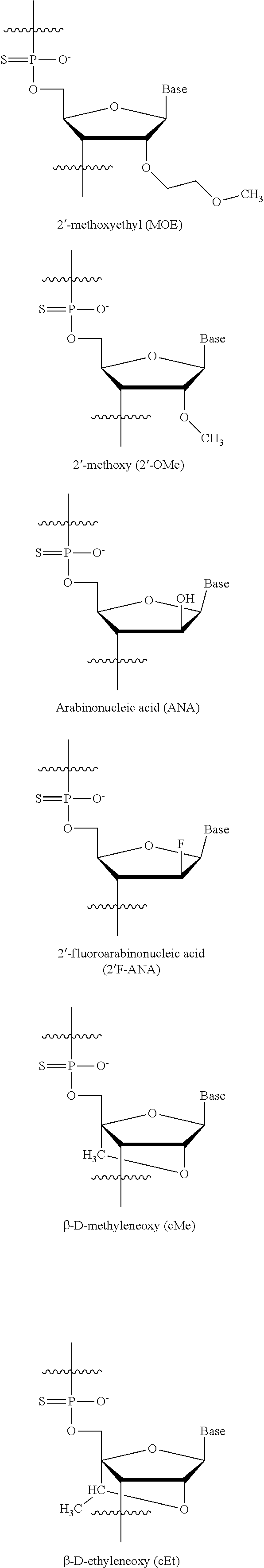

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854).

A single example of a gapmer compound of the present invention is gapmer compound number 223 (SEQ ID NO: 223), which comprises a 5′ wing and 3′ wing segment of modified nucleosides each having four 2′-methoxyethyl (MOE) modifications, and a central gap region sequence having ten 2′-deoxynucleosides, and wherein the linked nucleosides are linked with phosphorothioate internucleoside linkages. The modification sequence for gapmer compound 223 is “MMMMddddddddddMMMM”, where “M” is the 2′-methoxyethyl (MOE) modification, and “d” is an unmodified deoxyribose. The base sequence for gapmer compound 223 is TTCTTGAGCAGTAATTCA, and the structure is shown below, where “connection ‘A’ and connection ‘B’ indicates how the three fragments shown are connected together.

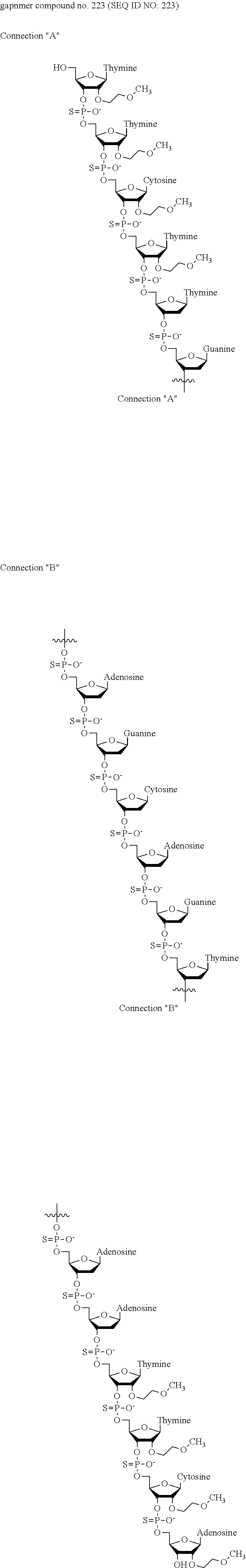

Administration

The gapmers described herein may be administered in a number of ways depending upon whether local or systemic treatment is desired and upon the area to be treated. Administration may be topical, pulmonary, e.g., by inhalation or insufflation of powders or aerosols, including by nebulizer; intratracheal, intraocular, intranasal, epidermal and transdermal, oral or parenteral. The compounds and compositions described herein can be delivered in a manner to target a particular tissue, such as the eye, bone marrow or brain. The compounds and compositions described herein are administered parenterally. “Parenteral administration” means administration through injection or infusion. Parenteral administration includes subcutaneous administration, intravenous administration, intraocular administration, intramuscular administration, intraarterial administration, intraperitoneal administration, or intracranial administration, e.g. intracerebral administration, intrathecal administration, intraventricular administration, ventricular administration, intracerebroventricular administration, cerebral intraventricular administration or cerebral ventricular administration. Administration can be continuous, or chronic, or short or intermittent.

Parenteral administration is also by infusion. Infusion can be chronic or continuous or short or intermittent, with a pump or by injection. Or parenteral administration is subcutaneous.

Such compositions comprise a pharmaceutically acceptable solvent, such as water or saline, diluent, carrier, or adjuvant. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial (intrathecal or intraventricular, administration).

The gapmer compounds may also be admixed, conjugated or otherwise associated with other molecules, molecule structures or mixtures of compounds, as for example, liposomes, receptor-targeted molecules, or other formulations, for assisting in uptake, distribution and/or absorption.

The gapmer compounds include any pharmaceutically acceptable carriers, esters, or carriers of such esters, or any other compound which, upon administration to an animal, including a human, is capable of providing (directly or indirectly) the biologically active metabolite or residue thereof.

The term “pharmaceutically acceptable excipients, carriers or diluents” refers to physiologically and pharmaceutically acceptable excipients, carriers, or diluents of the gapmer compounds i.e., carriers that retain the desired biological activity of the parent compound and do not impart undesired toxicological effects thereto. For gapmer compounds of the present disclosure, preferred examples of pharmaceutically acceptable carriers and their uses are further described in U.S. Pat. No. 6,287,860, which is incorporated by reference herein. Sodium carriers have been shown to be suitable forms of oligonucleotide drugs.

Formulations include liposomal formulations. The term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Liposomes and their uses are further described in U.S. Pat. No. 6,287,860, which is incorporated herein.

Preferred formulations for topical administration include those in which the oligonucleotides are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

Lipid Nanoparticles

LNPs are multi-component systems that typically consist of an ionizable amino lipid, a phospholipid, cholesterol, and a polyethylene glycol (PEG)-lipid, with all of the components contributing to efficient delivery of the nucleic acid drug cargo and stability of the particle (Schroeder et al., Lipid-based nanotherapeutics for siRNA delivery. J. Intern. Med. 2010; 267:9-21). The cationic lipid electrostatically condenses the negatively charged RNA into nanoparticles and the use of ionizable lipids that are positively charged at acidic pH is thought to enhance endosomal escape. Formulations for delivery of siRNA, both clinically and non-clinically, are predominantly based on cationic lipids such as DLin-MC3-DMA (MC3). (Kanasty et al. “Delivery materials for siRNA therapeutics.” Nat. Mater. 2013; 12:967-977; and Xue et al. “Lipid-based nanocarriers for RNA delivery.” Curr. Pharm. Des. 2015; 21:3140-3147).

Further LNP's include a nanoemulsion having a perfluorcarbon component (a) consisting of at least one least one perfluorcarbon compound, an emulsifying component (b) such as phospholipids and optionally helper lipids, and an endocytosis enhancing component (c) that comprises at least one compound inducing cellular uptake of the nanoemulsion. A perfluorcarbon compound of component (a) is preferably selected from compounds having the structure CₘF₂ₘ₊₁X, XCₘF₂ₘX, XCₙF₂ₙOCₒF₂ₒX, N(CₒF₂ₒX)₃ and N(CₒF₂ₒ₊₁)₃, wherein m is an integer from 3 to 10, n and o are integers from 1 to 5, and X is independently from further occurrence selected from Cl, Br and I. Examples of perfluorcarbon compounds are perfluorooctyl bromide and perfluorotributylamine.

Examples of the emulsifying agents include phospholipids, such as the phospholipid compound represented by the formula I:

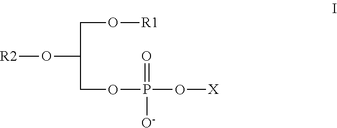

wherein
R¹ and R² are independently selected from H and C₁₆₋₂₄ acyl residues, which may be saturated or unsaturated and may carry 1 to 3 residues R³ and wherein one or more of the C-atoms may be substituted by O or NR⁴, and
X is selected from H, —(CH₂)ₚ—N(R⁴)₃⁺, —(CH₂)ₚ—CH(N(R⁴)₃⁺)—COO⁻, —(CH₂)ₚ—CH(OH)—CH₂OH and —CH₂(CHOH)ₚ—CH₂OH (wherein p is an integer from 1 to 5;
R³ is independently selected from H, lower alkyl, F, Cl, CN und OH; and
R⁴ is independently selected from H, CH₃ und CH₂CH₃, or a pharmacologically acceptable carrier thereof.

Following subcutaneous (s.c.) administration, LNPs and their mRNA cargo are expected to be largely retained at the site of injection, resulting in high local concentrations. Since LNPs are known to be pro-inflammatory, largely attributed to the ionizable lipid present in the LNPs, (Sabnis et al. “A novel amino lipid series for mRNA delivery: improved endosomal escape and sustained pharmacology and safety in non-human primates.” Mol. Ther. 2018; 26:1509-1519) then it would not be unexpected that s.c. administration of mRNA formulated in LNPs would be associated with dose-limiting inflammatory responses. Co-administration of dexamethasone with LNP reduces the immune-inflammatory response following i.v. administration (Abrams et al. “Evaluation of efficacy, biodistribution, and inflammation for a potent siRNA nanoparticle: Effect of dexamethasone co-treatment.” Mol. Ther. 2010; 18:171-180). And Chen et al. (“Dexamethasone prodrugs as potent suppressors of the immunostimulatory effects of lipid nanoparticle formulations of nucleic acids.” J. Control. Release. 2018; 286:46-54.) showed reduced immune stimulation following systemic administration by incorporating lipophilic dexamethasone prodrugs within LNP-containing nucleic acids.

Dosing

Optimal dosing schedules are calculated from measurements of drug accumulation in the body of the patient. Optimum dosages vary depending on the relative potency of individual gapmer compounds, and can generally be estimated based on EC₅₀s found to be effective in in vitro and in vivo animal models. In general, dosage is from 0.01 μg to 100 g per kg of body weight, and may be given once or more daily, weekly, monthly or yearly, or at desired intervals. Following successful treatment, it may be desirable to have the patient undergo maintenance therapy to prevent the recurrence of the disease state, wherein the gapmer compound is administered in maintenance doses, ranging from 0.01 μg to 100 g per kg of body weight, once or more daily.

### EXAMPLES

Additional embodiments are disclosed in further detail in the following examples, which are not intended to limit the scope of the claims.

### Example 1

This example provides a screening system for in vitro assays of candidate Gapmers for inhibiting gene transcription regulated by long non-coding RNA UMLILO.

Cell Culture and Oligonucleotide Treatment:

The effect of gapmer compounds were screened for target nucleic acid expression (e.g., messenger RNA) by RT-PCR.

THP-1 Cells

THP-1 human monocytic cell line (derived from an acute leukaemia patient) was obtained from InvivoGen. THP1 cells were maintained in complete media which is composed of RPMI 1640, 1% (2 mM) GlutaMAX L-glutamine supplement, 25 mM HEPES, 10% FBS, 100 μg/ml Normocin, Pen-Strep (100 U/ml), Blasticidin (10 μg/ml) and Zeocin (100 μg/ml).

Treatment with Antisense Compounds:

Prior to seeding for the screen, the THP-1 monocyte culture was split by 50% to enable the cells to re-enter an exponential growth phase. 250,000 cells were seeded per well in quadruplicate in 96-well plates with 180 μL of complete medium in each well. Each gapmer compound tested was added to the THP-1 cells at a final concentration of 10 μM and mixed gently. Plates were incubated at 37° C. at 5% CO₂ for 24 hours. Then, LPS (10 ng/mL) was added to each well, and plates were incubated at 37° C. at 5% CO₂ for another 24 hours.

Analysis of Oligonucleotide Inhibition of UMLILO Expression:

Antisense modulation of UMLILO expression on specified genes was assayed by real-time PCR (RT-PCR).

RNA analysis was performed on total cellular RNA or poly(A)+ mRNA. RNA was isolated and prepared using TRIZOL® Reagent (ThermoFisher Scientific) and Direct-zol RNA Miniprep Kit (Zymo Research) according to the manufacturer's recommended protocols.

Real-Time Quantitative PCR Analysis of mRNA Levels:

Quantitation of target RNA levels was accomplished by quantitative real-time PCR using, a CFX Real-time qPCR detection system (Biorad). Prior to real-time PCR, the isolated RNA was subjected to a reverse transcriptase (RT) reaction, which produces complementary DNA (cDNA) that is then used as the substrate for the real-time PCR amplification. RT reaction reagents and real-time PCR reagents were obtained from ThermoFisher Scientific, and protocols for their use are provided by the manufacturer. Gene (or RNA) target quantities obtained by real time PCR were normalized using expression levels of a gene whose expression is constant, such as HPRT or RPL37A. Total RNA was quantified using a Qubit Fluorometer (Invitrogen/ThermoFischer Scientific) and a Qubit RNA HS Assay Kit (ThermoFisher Scientific Cat. No. Q32852) in accordance with the manufacturer's protocol. The Qubit Flourometer was calibrated with standards.

A series of gapmer compounds of the present disclosure were designed to target different regions of the human UMLILO lnc RNA (Ensembl Gene ID: ENSG00000228277) (SEQ ID NO: 231). The compounds are shown in Table 1. The gapmer compounds in Table 1 are chimeric oligonucleotides (“gapmer compounds”) having a configuration of: a) 20 (5-10-5) nucleotides in length, composed of a central “gap” region comprising ten 2′-deoxynucleotides, which is flanked on both sides (5′ and 3′ directions) by five-nucleotide “wings”. In some example gapmer compounds, the wings were composed of 2′-methoxyethyl (2′-MOE) sugar modified nucleosides The internucleotide (backbone) linkages were phosphorothioate throughout the entire oligonucleotide sequence. Cytidine residues were 5-methylcytidines unless indicated otherwise, in which case they were cytidines residues; or b) 16 (3-10-3) nucleotides in length, composed of a central “gap” region comprising ten 2′-deoxynucleotides, which was flanked on both sides (5′ and 3′ directions) by three-nucleotide “wings”. Other configurations and modified nucleosides of the wing segments are shown in Table 1. In some cases, the wings were composed of locked nucleic acid (LNA) modified nucleosides employing the cMe locked nucleic acid modification. The internucleotide (backbone) linkages were phosphorothioate throughout the entire oligonucleotide sequence. Cytidine residues were 5-methylcytidines unless indicated otherwise, in which case they were cytidines residues.

Table 1 describe a group of 297 gapmer compounds that were synthesized and tested.

Oligonucleotide and Oligonucleoside Synthesis

The antisense compounds are made by solid phase synthesis by phosphorothioates and alkylated derivatives. Equipment for such synthesis is sold by several vendors including, for example, KareBay Bio (New Jersey, USA). Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Design and Screening of Duplexed Antisense Compounds Targeting UMLILO

Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, CA, or Pharmacia, Piscataway, NJ). Non-standard nucleosides are synthesized as per standard or patented methods. They are utilized as base protected beta-cyanoethyldiisopropyl phosphoramidites

Oligonucleotides were cleaved from support and deprotected with concentrated NH4OH at elevated temperature (55-60° C.) for 12-16 hours and the released product then dried in a vacuum. The dried product was then re-suspended in sterile water to afford a master plate from which all analytical and test plate samples are then diluted utilizing robotic pipettors.

TABLE 1
Gapmer compounds used in the present examples and embodiments
described herein. Abbreviations for Table 1: Nucleoside modification chemistry: M = 2′-
methoxyethyl (2′-MOE) modified nucleoside; 2′M = 2′OMe modified nucleoside; C = cET
modified LNA nucleoside; L = cMe modified LNA nucleoside; and d = 2′-deoxynucleosides.

| Gapmer | | | | | Complementary |
|---|---|---|---|---|---|
| Com- | SEQ ID | | Nucleoside | Sequence of | to human |
| pound No. | NO: | Configuration | Modification | gapmer compound | UMLILO position |
| 1 | 1 | 5-10-5 | MMMMMddddd | CATTTCCAACTT | 132→151 |
| | | | dddddMMMMM | GTAGGGTT | |
| 2 | 2 | 5-10-5 | MMMMMddddd | GTGTTTGAGAGG | 147→166 |
| | | | dddddMMMMM | AGCCATTT | |
| 3 | 3 | 5-10-5 | MMMMMddddd | AGTGTTTGAGAG | 148→167 |
| | | | dddddMMMMM | GAGCCATT | |
| 4 | 4 | 5-10-5 | MMMMMddddd | GTAGCAGATACA | 179→198 |
| | | | dddddMMMMM | TTTGGGCT | |
| 5 | 5 | 5-10-5 | MMMMMddddd | AGTAGCAGATAC | 180→199 |
| | | | dddddMMMMM | ATTTGGGC | |
| 6 | 6 | 5-10-5 | MMMMMddddd | TTTTGCATTATG | 203→222 |
| | | | dddddMMMMM | GAAGGCAC | |
| 7 | 7 | 5-10-5 | MMMMMddddd | GTTTTGCATTAT | 204→223 |
| | | | dddddMMMMM | GGAAGGCA | |
| 8 | 8 | 5-10-5 | MMMMMddddd | CAATTTTGGTAG | 245→264 |
| | | | dddddMMMMM | CAGTAGAC | |
| 9 | 9 | 5-10-5 | MMMMMddddd | GGTTACAATTTT | 250→269 |
| | | | dddddMMMMM | GGTAGCAG | |
| 10 | 10 | 5-10-5 | MMMMMddddd | GATGGGGGTTAC | 256→275 |
| | | | dddddMMMMM | AATTTTGG | |
| 11 | 11 | 5-10-5 | MMMMMddddd | TGATGGGGGTTA | 257→276 |
| | | | dddddMMMMM | CAATTTTG | |
| 12 | 12 | 5-10-5 | MMMMMddddd | TACTAGATGATG | 264→283 |
| | | | dddddMMMMM | GGGGTTAC | |
| 13 | 13 | 5-10-5 | MMMMMddddd | TTACTAGATGAT | 265→284 |
| | | | dddddMMMMM | GGGGGTTA | |
| 14 | 14 | 5-10-5 | MMMMMddddd | CTGGAATGAAGC | 319→338 |
| | | | dddddMMMMM | AGAATACA | |
| 15 | 15 | 5-10-5 | MMMMMddddd | TCTGGAATGAAG | 320→339 |
| | | | dddddMMMMM | CAGAATAC | |
| 16 | 16 | 5-10-5 | MMMMMddddd | AGGCCTCTGGAA | 325→344 |
| | | | dddddMMMMM | TGAAGCAG | |
| 17 | 17 | 5-10-5 | MMMMMddddd | AGTATTCTATGT | 375→394 |
| | | | dddddMMMMM | GAGCAGCT | |
| 18 | 18 | 5-10-5 | MMMMMddddd | AAGTATTCTATG | 376→395 |
| | | | dddddMMMMM | TGAGCAGC | |
| 19 | 19 | 5-10-5 | MMMMMddddd | ACTGGTGAAAAT | 400→419 |
| | | | dddddMMMMM | GGAAGTCA | |
| 20 | 20 | 5-10-5 | MMMMMddddd | AACTGGTGAAA | 401→420 |
| | | | dddddMMMMM | ATGGAAGTC | |
| 21 | 21 | 5-10-5 | MMMMMddddd | TTACAGACTATA | 521→540 |
| | | | dddddMMMMM | CAGTTTAG | |
| 22 | 22 | 5-10-5 | MMMMMddddd | TTTACAGACTAT | 522→541 |
| | | | dddddMMMMM | ACAGTTTA | |
| 23 | 23 | 5-10-5 | MMMMMddddd | CAGACTATACAG | 518→537 |
| | | | dddddMMMMM | TTTAGTAG | |
| 24 | 24 | 5-10-5 | MMMMMddddd | ACAGACTATACA | 519→538 |
| | | | dddddMMMMM | GTTTAGTA | |
| 25 | 25 | 5-10-5 | MMMMMddddd | TACAGACTATAC | 520→539 |
| | | | dddddMMMMM | AGTTTAGT | |
| 26 | 26 | 5-10-5 | MMMMMddddd | CTTTACAGACTA | 523→542 |
| | | | dddddMMMMM | TACAGTTT | |
| 27 | 27 | 5-10-5 | MMMMMddddd | CCTTTACAGACT | 524→543 |
| | | | dddddMMMMM | ATACAGTT | |
| 28 | 28 | 5-10-5 | MMMMMddddd | GACTGAGATAA | 547→566 |
| | | | dddddMMMMM | ATACAGTTC | |
| 29 | 29 | 5-10-5 | MMMMMddddd | TGACTGAGATAA | 548→567 |
| | | | dddddMMMMM | ATACAGTT | |
| 30 | 30 | 5-10-5 | MMMMMddddd | ATACAGTTTAGT | 512→531 |
| | | | dddddMMMMM | AGACTCAA | |
| 31 | 31 | 5-10-5 | MMMMMddddd | TGCCTTTACAGA | 526→545 |
| | | | dddddMMMMM | CTATACAG | |
| 32 | 32 | 5-10-5 | MMMMMddddd | GCCTTTACAGAC | 525→544 |
| | | | dddddMMMMM | TATACAGT | |
| 33 | 33 | 5-10-5 | MMMMMddddd | CTGCCTTTACAG | 527→546 |
| | | | dddddMMMMM | ACTATACA | |
| 34 | 34 | 5-10-5 | MMMMMddddd | TATACAGTTTAG | 513→532 |
| | | | dddddMMMMM | TAGACTCA | |
| 35 | 35 | 5-10-5 | MMMMMddddd | CCTGCCTTTACA | 528→547 |
| | | | dddddMMMMM | GACTATAC | |
| 36 | 36 | 5-10-5 | MMMMMddddd | ACTTCTTGAGCA | 444→463 |
| | | | dddddMMMMM | GTAATTCA | |
| 37 | 37 | 5-10-5 | MMMMMddddd | CTTCTTGAGCAG | 443→462 |
| | | | dddddMMMMM | TAATTCAG | |
| 38 | 38 | 5-10-5 | MMMMMddddd | TTCTTGAGCAGT | 442→461 |
| | | | dddddMMMMM | AATTCAGC | |
| 39 | 39 | 5-10-5 | MMMMMddddd | TACTTCTTGAGC | 445→464 |
| | | | dddddMMMMM | AGTAATTC | |
| 40 | 40 | 5-10-5 | MMMMMddddd | TCTTGAGCAGTA | 441→460 |
| | | | dddddMMMMM | ATTCAGCA | |
| 41 | 41 | 5-10-5 | MMMMMddddd | TCCTGCCTTTAC | 529→548 |
| | | | dddddMMMMM | AGACTATA | |
| 42 | 42 | 5-10-5 | MMMMMddddd | AAGGCATGTGG | 461→480 |
| | | | dddddMMMMM | ATCTATACT | |
| 43 | 43 | 5-10-5 | MMMMMddddd | CTTGAGCAGTAA | 440→459 |
| | | | dddddMMMMM | TTCAGCAT | |
| 44 | 44 | 5-10-5 | MMMMMddddd | AGGCATGTGGAT | 460→479 |
| | | | dddddMMMMM | CTATACTT | |
| 45 | 45 | 5-10-5 | MMMMMddddd | TTGAGCAGTAAT | 439→458 |
| | | | dddddMMMMM | TCAGCATC | |
| 46 | 46 | 5-10-5 | MMMMMddddd | CTGAAGTTGAAG | 470→489 |
| | | | dddddMMMMM | GCATGTGG | |
| 47 | 47 | 5-10-5 | MMMMMddddd | TCTGAAGTTGAA | 471→490 |
| | | | dddddMMMMM | GGCATGTG | |
| 48 | 48 | 5-10-5 | MMMMMddddd | TTCTGAAGTTGA | 472→491 |
| | | | dddddMMMMM | AGGCATGT | |
| 49 | 49 | 5-10-5 | MMMMMddddd | TTTAAGATTCTG | 479→498 |
| | | | dddddMMMMM | AAGTTGAA | |
| 50 | 50 | 5-10-5 | MMMMMddddd | ATGTGGATCTAT | 456→475 |
| | | | dddddMMMMM | ACTTCTTG | |
| 51 | 51 | 5-10-5 | MMMMMddddd | TGTGGATCTATA | 455→474 |
| | | | dddddMMMMM | CTTCTTGA | |
| 52 | 52 | 5-10-5 | MMMMMddddd | TGAAGGCATGTG | 463→482 |
| | | | dddddMMMMM | GATCTATA | |
| 53 | 53 | 5-10-5 | MMMMMddddd | TTGAAGGCATGT | 464→483 |
| | | | dddddMMMMM | GGATCTAT | |
| 54 | 54 | 5-10-5 | MMMMMddddd | GTGGATCTATAC | 454→473 |
| | | | dddddMMMMM | TTCTTGAG | |
| 55 | 55 | 5-10-5 | MMMMMddddd | TCTATACTTCTT | 449→468 |
| | | | dddddMMMMM | GAGCAGTA | |
| 56 | 56 | 5-10-5 | MMMMMddddd | ATCTATACTTCT | 450→469 |
| | | | dddddMMMMM | TGAGCAGT | |
| 57 | 57 | 5-10-5 | MMMMMddddd | TGGATCTATACT | 453→472 |
| | | | dddddMMMMM | TCTTGAGC | |
| 58 | 58 | 5-10-5 | MMMMMddddd | ACAGTTCCTGCC | 534→553 |
| | | | dddddMMMMM | TTTACAGA | |
| 59 | 59 | 5-10-5 | MMMMMddddd | CTATACTTCTTG | 448→467 |
| | | | dddddMMMMM | AGCAGTAA | |
| 60 | 60 | 5-10-5 | MMMMMddddd | GGATCTATACTT | 452→471 |
| | | | dddddMMMMM | CTTGAGCA | |
| 61 | 61 | 5-10-5 | MMMMMddddd | TATACTTCTTGA | 447→466 |
| | | | dddddMMMMM | GCAGTAAT | |
| 62 | 62 | 5-10-5 | MMMMMddddd | GATCTATACTTC | 451→470 |
| | | | dddddMMMMM | TTGAGCAG | |
| 63 | 63 | 5-10-5 | MMMMMddddd | TGAGCAGTAATT | 438→457 |
| | | | dddddMMMMM | CAGCATCT | |
| 64 | 64 | 3-10-3 | LLLdddddddddd | CTGGAGTGTTGC | 79→94 |
| | | | LLL | TGCG | |
| 65 | 65 | 3-10-3 | LLLdddddddddd | TGTTCACCGTGT | 290→305 |
| | | | LLL | ACCA | |
| 66 | 66 | 3-10-3 | LLLdddddddddd | GGGGTTACAATT | 256→271 |
| | | | LLL | TTGG | |
| 67 | 67 | 3-10-3 | LLLdddddddddd | TATGCAGACTAA | 114→129 |
| | | | LLL | AAAA | |
| 68 | 68 | 3-10-3 | LLLdddddddddd | TTAAGATTCTGA | 482→497 |
| | | | LLL | AGTT | |
| 69 | 69 | 3-10-3 | LLLdddddddddd | CAGACTAAAAA | 110→125 |
| | | | LLL | AGATC | |
| 70 | 70 | 3-10-3 | LLLdddddddddd | CCACACATAAA | 95→110 |
| | | | LLL | GCGCC | |
| 71 | 71 | 3-10-3 | LLLdddddddddd | CTCAACATTCGC | 501→516 |
| | | | LLL | CTCT | |
| 72 | 72 | 3-10-3 | LLLdddddddddd | TTTACAGACTAT | 526→541 |
| | | | LLL | ACAG | |
| 73 | 73 | 3-10-3 | LLLdddddddddd | ACTATACAGTTT | 519→534 |
| | | | LLL | AGTA | |
| 74 | 74 | 3-10-3 | LLLdddddddddd | CTATACAGTTTA | 518→533 |
| | | | LLL | GTAG | |
| 75 | 75 | 3-10-3 | LLLdddddddddd | AGTTCCTGCCTT | 536→551 |
| | | | LLL | TACA | |
| 76 | 76 | 3-10-3 | LLLdddddddddd | TAAAGCGCCCTG | 88→103 |
| | | | LLL | GAGT | |
| 77 | 77 | 3-10-3 | LLLdddddddddd | GCTGCGAGAATT | 69→84 |
| | | | LLL | TGTA | |
| 78 | 78 | 3-10-3 | LLLdddddddddd | AGATGAACTGTT | 44→59 |
| | | | LLL | ATGA | |
| 79 | 79 | 3-10-3 | LLLdddddddddd | TTACTAGATGAT | 269→284 |
| | | | LLL | GGGG | |
| 80 | 80 | 3-10-3 | LLLdddddddddd | TAATTTAAGATT | 486→501 |
| | | | LLL | CTGA | |
| 81 | 81 | 3-10-3 | LLLdddddddddd | TACTAGATGATG | 268→283 |
| | | | LLL | GGGG | |
| 82 | 82 | 3-10-3 | LLLdddddddddd | ATGCAGACTAAA | 113→128 |
| | | | LLL | AAAG | |
| 83 | 83 | 3-10-3 | LLLdddddddddd | ACATTCGCCTCT | 497→512 |
| | | | LLL | AATT | |
| 84 | 84 | 3-10-3 | LLLdddddddddd | TACAGACTATAC | 524→539 |
| | | | LLL | AGTT | |
| 85 | 85 | 3-10-3 | LLLdddddddddd | GACTGAGATAA | 551→566 |
| | | | LLL | ATACA | |
| 86 | 86 | 3-10-3 | LLLdddddddddd | TATACAGTTTAG | 517→532 |
| | | | LLL | TAGA | |
| 87 | 87 | 3-10-3 | LLLdddddddddd | GTTCCTGCCTTT | 535→550 |
| | | | LLL | ACAG | |
| 88 | 88 | 3-10-3 | LLLdddddddddd | GCAGGCCTCTGG | 331→346 |
| | | | LLL | AATG | |
| 89 | 89 | 3-10-3 | LLLdddddddddd | TTGGGCTGCTCT | 170→185 |
| | | | LLL | ATCT | |
| 90 | 90 | 3-10-3 | LLLdddddddddd | CTGATCTAAACT | 413→428 |
| | | | LLL | GGTG | |
| 91 | 91 | 3-10-3 | LLLdddddddddd | TTAACACAGATG | 51→66 |
| | | | LLL | AACT | |
| 92 | 92 | 3-10-3 | LLLdddddddddd | CTAATTTAAGAT | 487→502 |
| | | | LLL | TCTG | |
| 93 | 93 | 3-10-3 | LLLdddddddddd | ACTCTTTACTAG | 274→289 |
| | | | LLL | ATGA | |
| 94 | 94 | 3-10-3 | LLLdddddddddd | TCTTTACTAGAT | 272→287 |
| | | | LLL | GATG | |
| 95 | 95 | 3-10-3 | LLLdddddddddd | TAGACTCAACAT | 505→520 |
| | | | LLL | TCGC | |
| 96 | 96 | 3-10-3 | LLLdddddddddd | CTTTACAGACTA | 527→542 |
| | | | LLL | TACA | |
| 97 | 97 | 3-10 -3 | LLLdddddddddd | ACTGAGATAAAT | 550→565 |
| | | | LLL | ACAG | |
| 98 | 98 | 3-10-3 | LLLdddddddddd | TTACAGACTATA | 525→540 |
| | | | LLL | CAGT | |
| 99 | 99 | 3-10-3 | LLLdddddddddd | TAATCTCCACAT | 1→16 |
| | | | LLL | GTAT | |
| 100 | 100 | 3-10-3 | LLLdddddddddd | CACATAAAGCG | 92→107 |
| | | | LLL | CCCTG | |
| 101 | 101 | 3-10-3 | LLLdddddddddd | GCATTATGGAAG | 203→218 |
| | | | LLL | GCAC | |
| 102 | 102 | 3-10-3 | LLLdddddddddd | GAGATTAAGAAT | 345→360 |
| | | | LLL | TGGC | |
| 103 | 103 | 3-10-3 | LLLdddddddddd | GTTAACACAGAT | 52→67 |
| | | | LLL | GAAC | |
| 104 | 104 | 3-10-3 | LLLdddddddddd | CCTCTAATTTAA | 490→505 |
| | | | LLL | GATT | |
| 105 | 105 | 3-10-3 | LLLdddddddddd | CTATGTGAGCAG | 373→388 |
| | | | LLL | CTCT | |
| 106 | 106 | 3-10-3 | LLLdddddddddd | AAGATTCTGAAG | 480→495 |
| | | | LLL | TTGA | |
| 107 | 107 | 3-10-3 | LLLdddddddddd | AGGCACCACAG | 193→208 |
| | | | LLL | TAGCA | |
| 108 | 108 | 3-10-3 | LLLdddddddddd | TTGCATTATGGA | 205→220 |
| | | | LLL | AGGC | |
| 109 | 109 | 3-10-3 | LLLdddddddddd | TCCACTGATCTA | 417→432 |
| | | | LLL | AACT | |
| 110 | 110 | 3-10-3 | LLLdddddddddd | GAGTGTTGCTGC | 76→91 |
| | | | LLL | GAGA | |
| 111 | 111 | 3-10-3 | LLLdddddddddd | TGTTAACACAGA | 53→68 |
| | | | LLL | TGAA | |
| 112 | 112 | 3-10-3 | LLLdddddddddd | AGATTCTGAAGT | 479→494 |
| | | | LLL | TGAA | |
| 113 | 113 | 3-10-3 | LLLdddddddddd | ATAAAGCGCCCT | 89→104 |
| | | | LLL | GGAG | |
| 114 | 114 | 3-10-3 | LLLdddddddddd | GGGGGTTACAAT | 257→272 |
| | | | LLL | TTTG | |
| 115 | 115 | 3-10-3 | LLLdddddddddd | AGACTATACAGT | 521→536 |
| | | | LLL | TTAG | |
| 116 | 116 | 3-10-3 | LLLdddddddddd | TGACTGAGATAA | 552→567 |
| | | | LLL | ATAC | |
| 117 | 117 | 3-10-3 | LLLdddddddddd | TTCAGCATCTCT | 432→447 |
| | | | LLL | CTGT | |
| 118 | 118 | 3-10-3 | LLLdddddddddd | CTTAATCTCCAC | 3→18 |
| | | | LLL | ATGT | |
| 119 | 119 | 3-10-3 | LLLdddddddddd | TATGTGAGCAGC | 372→387 |
| | | | LLL | TCTT | |
| 120 | 120 | 3-10-3 | LLLdddddddddd | CTAGATGATGGG | 266→281 |
| | | | LLL | GGTT | |
| 121 | 121 | 3-10-3 | LLLdddddddddd | ATAAATACAGTT | 544→559 |
| | | | LLL | CCTG | |
| 122 | 122 | 3-10-3 | LLLdddddddddd | TTTACTAGATGA | 270→285 |
| | | | LLL | TGGG | |
| 123 | 123 | 3-10-3 | LLLdddddddddd | GCTGCGAGAATT | 69→84 |
| | | | LLL | TGTA | |
| 124 | 124 | 3-10-3 | LLLdddddddddd | TTTAAGATTCTG | 483→498 |
| | | | LLL | AAGT | |
| 125 | 125 | 3-10-3 | LLLdddddddddd | AACTTGTAGGGT | 129→144 |
| | | | LLL | TAAT | |
| 126 | 126 | 3-10-3 | LLLdddddddddd | TTGTAGGGTTAA | 126→141 |
| | | | LLL | TATG | |
| 127 | 127 | 3-10-3 | LLLdddddddddd | CAGACTATACAG | 522→537 |
| | | | LLL | TTTA | |
| 128 | 128 | 3-10-3 | LLLdddddddddd | TGAGATAAATAC | 548→563 |
| | | | LLL | AGTT | |
| 129 | 129 | 3-10-3 | LLLdddddddddd | GTCTTAATCTCC | 5→20 |
| | | | LLL | ACAT | |
| 130 | 130 | 3-10-3 | LLLdddddddddd | GCAGTAATTCAG | 439→454 |
| | | | LLL | CATC | |
| 131 | 131 | 3-10-3 | LLLdddddddddd | TGAAGGCATGTG | 467→482 |
| | | | LLL | GATC | |
| 132 | 132 | 3-10-3 | LLLdddddddddd | AGTGTTTGAGAG | 152→167 |
| | | | LLL | GAGC | |
| 133 | 133 | 3-10-3 | LLLdddddddddd | AGTGTTGCTGCG | 75→90 |
| | | | LLL | AGAA | |
| 134 | 134 | 3-10-3 | LLLdddddddddd | CTTTACTAGATG | 271→286 |
| | | | LLL | ATGG | |
| 135 | 135 | 3-10-3 | LLLdddddddddd | TTGCTGCGAGAA | 71→86 |
| | | | LLL | TTTG | |
| 136 | 136 | 3-10-3 | LLLdddddddddd | ATTTAAGATTCT | 484→499 |
| | | | LLL | GAAG | |
| 137 | 137 | 3-10-3 | LLLdddddddddd | CCGTGTACCAAC | 284→299 |
| | | | LLL | TCTT | |
| 138 | 138 | 3-10-3 | LLLdddddddddd | CTTGTAGGGTTA | 127→142 |
| | | | LLL | ATAT | |
| 139 | 139 | 3-10-3 | LLLdddddddddd | CCTTTACAGACT | 528→543 |
| | | | LLL | ATAC | |
| 140 | 140 | 3-10-3 | LLLdddddddddd | TACTTCTTGAGC | 449→464 |
| | | | LLL | AGTA | |
| 141 | 141 | 3-10-3 | LLLdddddddddd | CAGTTCCTGCCT | 537→552 |
| | | | LLL | TTAC | |
| 142 | 142 | 3-10-3 | LLLdddddddddd | CAGTAATTCAGC | 438→453 |
| | | | LLL | ATCT | |
| 143 | 143 | 3-10-3 | LLLdddddddddd | TGCATTATGGAA | 204→219 |
| | | | LLL | GGCA | |
| 144 | 144 | 3-10-3 | LLLdddddddddd | AGACTCAACATT | 504→519 |
| | | | LLL | CGCC | |
| 145 | 145 | 3-10-3 | LLLdddddddddd | GTGTTGCTGCGA | 74→89 |
| | | | LLL | GAAT | |
| 146 | 146 | 3-10-3 | LLLdddddddddd | GATTCTGAAGTT | 478→493 |
| | | | LLL | GAAG | |
| 147 | 147 | 3-10-3 | LLLdddddddddd | TGTTGCTGCGAG | 73→88 |
| | | | LLL | AATT | |
| 148 | 148 | 3-10-3 | LLLdddddddddd | TGGAGTGTTGCT | 78→93 |
| | | | LLL | GCGA | |
| 149 | 149 | 3-10-3 | LLLdddddddddd | ACTCAACATTCG | 502→517 |
| | | | LLL | CCTC | |
| 150 | 150 | 3-10-3 | LLLdddddddddd | TCGCCTCTAATT | 493→508 |
| | | | LLL | TAAG | |
| 151 | 151 | 5-10-5 | MMMMMddddd | CTTCTTGAGCAG | 443→462 |
| | | | dddddMMMMM | TAATTCAG | |
| 152 | 152 | 6-8-6 | MMMMMMddd | CTTCTTGAGCAG | 443→462 |
| | | | dddddMMMMM | TAATTCAG | |
| | | | M | | |
| 153 | 153 | 7-6-7 | MMMMMMMd | CTTCTTGAGCAG | 443→462 |
| | | | dddddMMMMM | TAATTCAG | |
| | | | MM | | |
| 155 | 155 | 5-8-5 | MMMMMddddd | TTCTTGAGCAGT | 444→461 |
| | | | dddMMMMM | AATTCA | |
| 156 | 156 | 4-8-4 | MMMMddddddd | TCTTGAGCAGTA | 445→460 |
| | | | dMMMM | ATTC | |
| 157 | 157 | 5-6-5 | MMMMMddddd | TCTTGAGCAGTA | 445→460 |
| | | | dMMMMM | ATTC | |
| 158 | 158 | 3-10-4 | LLLdddddddddd | TCTTGAGCAGTA | 444→460 |
| | | | LLLL | ATTCA | |
| 159 | 159 | 5-8-4 | LLLLLdddddddd | TCTTGAGCAGTA | 444→460 |
| | | | LLLL | ATTCA | |
| 160 | 160 | 3-10-3 | LLLdddddddddd | TCTTGAGCAGTA | 445→460 |
| | | | LLL | ATTC | |
| 161 | 161 | 5-7-4 | LLLLLdddddddL | TCTTGAGCAGTA | 445→460 |
| | | | LLL | ATTC | |
| 162 | 162 | 3-10-3 | LLLd2′Mdddddd | TCTTGAGCAGTA | 445→460 |
| | | | ddLLL | ATTC | |
| 163 | 163 | 5-10-5 | MMMMMddddd | TACTAGATGATG | 264→283 |
| | | | dddddMMMMM | GGGGTTAC | |
| 164 | 164 | 6-8-6 | MMMMMMddd | TACTAGATGATG | 264→283 |
| | | | dddddMMMMM | GGGGTTAC | |
| | | | M | | |
| 165 | 165 | 7-6-7 | MMMMMMMd | TACTAGATGATG | 264→283 |
| | | | dddddMMMMM | GGGGTTAC | |
| | | | MM | | |
| 166 | 166 | 4-10-4 | MMMMddddddd | ACTAGATGATGG | 265→282 |
| | | | dddMMMM | GGGTTA | |
| 167 | 167 | 5-8-5 | MMMMMddddd | ACTAGATGATGG | 265→282 |
| | | | dddMMMMM | GGGTTA | |
| 168 | 168 | 4-8-4 | MMMMddddddd | CTAGATGATGGG | 266→281 |
| | | | dMMMM | GGTT | |
| 169 | 169 | 5-6-5 | MMMMMddddd | CTAGATGATGGG | 266→281 |
| | | | dMMMMM | GGTT | |
| 170 | 170 | 3-10-4 | LLLdddddddddd | CTAGATGATGGG | 265→281 |
| | | | LLLL | GGTTA | |
| 171 | 171 | 5-8-4 | LLLLLdddddddd | CTAGATGATGGG | 265→281 |
| | | | LLLL | GGTTA | |
| 172 | 172 | 3-10-3 | LLLdddddddddd | CTAGATGATGGG | 266→281 |
| | | | LLL | GGTT | |
| 173 | 173 | 5-7-4 | LLLLLdddddddL | CTAGATGATGGG | 266→281 |
| | | | LLL | GGTT | |
| 174 | 174 | 3-10-3 | LLLd2′Mdddddd | CTAGATGATGGG | 266→281 |
| | | | ddLLL | GGTT | |
| 175 | 175 | 5-10-5 | MMMMMddddd | CCTGCCTTTACA | 528→547 |
| | | | dddddMMMMM | GACTATAC | |
| 176 | 176 | 6-8-6 | MMMMMMddd | CCTGCCTTTACA | 528→547 |
| | | | dddddMMMMM | GACTATAC | |
| | | | M | | |
| 177 | 177 | 7-6-7 | MMMMMMMd | CCTGCCTTTACA | 528→547 |
| | | | dddddMMMMM | GACTATAC | |
| | | | MM | | |
| 178 | 178 | 4-10-4 | MMMMddddddd | CTGCCTTTACAG | 529→546 |
| | | | dddMMMM | ACTATA | |
| 179 | 179 | 5-8-5 | MMMMMddddd | CTGCCTTTACAG | 529→546 |
| | | | dddMMMMM | ACTATA | |
| 180 | 180 | 4-8-4 | MMMMddddddd | TGCCTTTACAGA | 530→545 |
| | | | dMMMM | CTAT | |
| 181 | 181 | 5-6-5 | MMMMMddddd | TGCCTTTACAGA | 530→545 |
| | | | dMMMMM | CTAT | |
| 182 | 182 | 3-10-4 | LLLdddddddddd | TGCCTTTACAGA | 529→545 |
| | | | LLLL | CTATA | |
| 183 | 183 | 5-8-4 | LLLLLdddddddd | TGCCTTTACAGA | 529→545 |
| | | | LLLL | CTATA | |
| 184 | 184 | 3-10-3 | LLLdddddddddd | TGCCTTTACAGA | 530→545 |
| | | | LLL | CTAT | |
| 185 | 185 | 5-7-4 | LLLLLdddddddL | TGCCTTTACAGA | 530→545 |
| | | | LLL | CTAT | |
| 186 | 186 | 3-10-3 | LLLd2′Mdddddd | TGCCTTTACAGA | 530→545 |
| | | | ddLLL | CTAT | |
| 187 | 187 | 5-10-5 | MMMMMddddd | TTACAGACTATA | 521→540 |
| | | | dddddMMMMM | CAGTTTAG | |
| 188 | 188 | 6-8-6 | MMMMMMddd | TTACAGACTATA | 521→540 |
| | | | dddddMMMMM | CAGTTTAG | |
| | | | M | | |
| 189 | 189 | 7-6-7 | MMMMMMMd | TTACAGACTATA | 521→540 |
| | | | dddddMMMMM | CAGTTTAG | |
| | | | MM | | |
| 190 | 190 | 4-10-4 | MMMMddddddd | TACAGACTATAC | 522→539 |
| | | | dddMMMM | AGTTTA | |
| 191 | 191 | 5-8-5 | MMMMMddddd | TACAGACTATAC | 522→539 |
| | | | dddMMMMM | AGTTTA | |
| 192 | 192 | 4-8-4 | MMMMddddddd | ACAGACTATACA | 523→538 |
| | | | dMMMM | GTTT | |
| 193 | 193 | 5-6-5 | MMMMMddddd | ACAGACTATACA | 523→538 |
| | | | dMMMMM | GTTT | |
| 194 | 194 | 3-10-4 | LLLdddddddddd | ACAGACTATACA | 522→538 |
| | | | LLLL | GTTTA | |
| 195 | 195 | 5-8-4 | LLLLLdddddddd | ACAGACTATACA | 522→538 |
| | | | LLLL | GTTTA | |
| 196 | 196 | 3-10-3 | LLLdddddddddd | CAGACTATACAG | 522→537 |
| | | | LLL | TTTA | |
| 197 | 197 | 5-7-4 | LLLLLdddddddL | CAGACTATACAG | 522→537 |
| | | | LLL | TTTA | |
| 198 | 198 | 3-10-3 | LLLd2′Mdddddd | CAGACTATACAG | 522→537 |
| | | | ddLLL | TTTA | |
| 199 | 199 | 5-10-5 | MMMMMddddd | CACACATAAAG | 90→109 |
| | | | dddddMMMMM | CGCCCTGGA | |
| 200 | 200 | 6-8-6 | MMMMMMddd | CACACATAAAG | 90→109 |
| | | | dddddMMMMM | CGCCCTGGA | |
| | | | M | | |
| 201 | 201 | 7-6-7 | MMMMMMMd | CACACATAAAG | 90→109 |
| | | | dddddMMMMM | CGCCCTGGA | |
| | | | MM | | |
| 202 | 202 | 4-10-4 | MMMMddddddd | ACACATAAAGC | 91→108 |
| | | | dddMMMM | GCCCTGG | |
| 203 | 203 | 5-8-5 | MMMMMddddd | ACACATAAAGC | 91→108 |
| | | | dddMMMMM | GCCCTGG | |
| 204 | 204 | 4-8-4 | MMMMddddddd | CACATAAAGCG | 92→107 |
| | | | dMMMM | CCCTG | |
| 205 | 205 | 5-6-5 | MMMMMddddd | CACATAAAGCG | 92→107 |
| | | | dMMMMM | CCCTG | |
| 206 | 206 | 3-10-4 | LLLdddddddddd | ACACATAAAGC | 92→108 |
| | | | LLLL | GCCCTG | |
| 207 | 207 | 5-8-4 | LLLLLdddddddd | ACACATAAAGC | 92→108 |
| | | | LLLL | GCCCTG | |
| 208 | 208 | 3-10-3 | LLLdddddddddd | CACATAAAGCG | 92→107 |
| | | | LLL | CCCTG | |
| 209 | 209 | 5-7-4 | LLLLLdddddddL | CACATAAAGCG | 92→107 |
| | | | LLL | CCCTG | |
| 210 | 210 | 3-10-3 | LLLd2′Mdddddd | CACATAAAGCG | 92→107 |
| | | | ddLLL | CCCTG | |
| 211 | 211 | 5-10-5 | MMMMMddddd | ACTGAGATAAAT | 546→565 |
| | | | dddddMMMMM | ACAGTTCC | |
| 212 | 212 | 6-8-6 | MMMMMMddd | ACTGAGATAAAT | 546→565 |
| | | | dddddMMMMM | ACAGTTCC | |
| | | | M | | |
| 213 | 213 | 7-6-7 | MMMMMMMd | ACTGAGATAAAT | 546→565 |
| | | | dddddMMMMM | ACAGTTCC | |
| | | | MM | | |
| 214 | 214 | 4-10-4 | MMMMddddddd | CTGAGATAAATA | 547→564 |
| | | | dddMMMM | CAGTTC | |
| 215 | 215 | 5-8-5 | MMMMMddddd | CTGAGATAAATA | 547→564 |
| | | | dddMMMMM | CAGTTC | |
| 216 | 216 | 4-8-4 | MMMMddddddd | TGAGATAAATAC | 548→563 |
| | | | dMMMM | AGTT | |
| 217 | 217 | 5-6-5 | MMMMMddddd | TGAGATAAATAC | 548→563 |
| | | | dMMMMM | AGTT | |
| 218 | 218 | 3-10-4 | LLLdddddddddd | CTGAGATAAATA | 548→564 |
| | | | LLLL | CAGTT | |
| 219 | 219 | 5-8-4 | LLLLLdddddddd | CTGAGATAAATA | 548→564 |
| | | | LLLL | CAGTT | |
| 221 | 221 | 5-7-4 | LLLLLdddddddL | TGAGATAAATAC | 548→563 |
| | | | LLL | AGTT | |
| 222 | 222 | 3-10 -3 | LLLd2′Mdddddd | TGAGATAAATAC | 548→563 |
| | | | ddLLL | AGTT | |
| 223 | 223 | 4-10-4 | MMMMddddddd | TTCTTGAGCAGT | 444→461 |
| | | | dddMMMM | AATTCA | |
| 224 | 224 | 4-10-4 | MMMMddddddd | TTCTTGAGCAGT | 444→461 |
| | | | dddMMMM | TATTCA | |
| 225 | 225 | 3-10-3 | LLLdddddddddd | CTTGAGCAGTAA | 444→459 |
| | | | LLL | TTCA | |
| 226 | 226 | 3-10-3 | LLLdddddddddd | CTTGAGCAGTTA | 444→459 |
| | | | LLL | TTCA | |
| 227 | 227 | 3-10-3 | LLLdd2′Mddddd | CTTGAGCAGTAA | 444→459 |
| | | | ddLLL | TTCA | |
| | 228 | | | AACACGTCTATA | None |
| | | | | CGC | |
| 230 | 230 | 3-10-3 | FFFdddddddddd | TCGCCTCTAATT | 444→461 |
| | | | FFF | TAAG | |
| 233 | 233 | 5-10-5 | MMMMMddddd | CTCTGGAATGAA | 321→340 |
| | | | dddddMMMMM | GCAGAATA | |
| 234 | 234 | 5-10-5 | MMMMMddddd | GCTCTATCTCCA | 159→178 |
| | | | dddddMMMMM | GTGTTTGA | |
| 235 | 235 | 5-10-5 | MMMMMddddd | GAATGAAGCAG | 316→335 |
| | | | dddddMMMMM | AATACATTC | |
| 236 | 236 | 5-10-5 | MMMMMddddd | ATTTGGGCTGCT | 168→187 |
| | | | dddddMMMMM | CTATCTCC | |
| 237 | 237 | 5-10-5 | MMMMMddddd | TGTGAGCAGCTC | 366→385 |
| | | | dddddMMMMM | TTCAGCCT | |
| 238 | 238 | 5-10-5 | MMMMMddddd | ACTTGTAGGGTT | 124→143 |
| | | | dddddMMMMM | AATATGCA | |
| 239 | 239 | 5-10-5 | MMMMMddddd | GCCTATAGTGAG | 350→369 |
| | | | dddddMMMMM | ATTAAGAA | |
| 240 | 240 | 5-10-5 | MMMMMddddd | TAGCAGATACAT | 178→197 |
| | | | dddddMMMMM | TTGGGCTG | |
| 241 | 241 | 5-10-5 | MMMMMddddd | CAGCCTATAGTG | 352→371 |
| | | | dddddMMMMM | AGATTAAG | |
| 242 | 242 | 5-10-5 | MMMMMddddd | AAACTGGTGAA | 402→421 |
| | | | dddddMMMMM | AATGGAAGT | |
| 243 | 243 | 5-10-5 | MMMMMddddd | CATTATGGAAGG | 198→217 |
| | | | dddddMMMMM | CACCACAG | |
| 244 | 244 | 5-10-5 | MMMMMddddd | ATGCAGACTAAA | 109→128 |
| | | | dddddMMMMM | AAAGATCC | |
| 245 | 245 | 5-10-5 | MMMMMddddd | CAGAATACATTC | 308→327 |
| | | | dddddMMMMM | CCACAGCA | |
| 246 | 246 | 5-10-5 | MMMMMddddd | CAACTTGTAGGG | 126→145 |
| | | | dddddMMMMM | TTAATATG | |
| 247 | 247 | 5-10-5 | MMMMMddddd | CAGAGAGTTTTG | 210→229 |
| | | | dddddMMMMM | CATTATGG | |
| 248 | 248 | 5-10-5 | MMMMMddddd | GAGCCATTTCCA | 136→155 |
| | | | dddddMMMMM | ACTTGTAG | |
| 249 | 249 | 5-10-5 | MMMMMddddd | CTAAAAAAGATC | 102→121 |
| | | | dddddMMMMM | CACACATA | |
| 250 | 250 | 5-10-5 | MMMMMddddd | GTAGACATATTC | 231→250 |
| | | | dddddMMMMM | TCAGCTCC | |
| 251 | 251 | 5-10-5 | MMMMMddddd | AAGGCACCACA | 190→209 |
| | | | dddddMMMMM | GTAGCAGAT | |
| 252 | 252 | 5-10-5 | MMMMMddddd | AGAGTTTTGCAT | 207→226 |
| | | | dddddMMMMM | TATGGAAG | |
| 253 | 253 | 5-10-5 | MMMMMddddd | TGGAAGGCACC | 193→212 |
| | | | dddddMMMMM | ACAGTAGCA | |
| 254 | 254 | 5-10-5 | MMMMMddddd | TCTCCAGTGTTT | 153→172 |
| | | | dddddMMMMM | GAGAGGAG | |
| 255 | 255 | 5-10-5 | MMMMMddddd | CTGCTCTATCTC | 161→180 |
| | | | dddddMMMMM | CAGTGTTT | |
| 256 | 256 | 5-10-5 | MMMMMddddd | AGATGAACTGTT | 40→59 |
| | | | dddddMMMMM | ATGAAAGT | |
| 257 | 257 | 5-10-5 | MMMMMddddd | TGAACTGTTATG | 37→56 |
| | | | dddddMMMMM | AAAGTGTT | |
| 258 | 258 | 5-10-5 | MMMMMddddd | GTCACTACAAGT | 384→403 |
| | | | dddddMMMMM | ATTCTATG | |
| 259 | 259 | 5-10-5 | MMMMMddddd | GAAGCAGAATA | 312→331 |
| | | | dddddMMMMM | CATTCCCAC | |
| 260 | 260 | 5-10-5 | MMMMMddddd | ATGAAGCAGAA | 314→333 |
| | | | dddddMMMMM | TACATTCCC | |
| 261 | 261 | 5-10-5 | MMMMMddddd | CAAGTATTCTAT | 377→396 |
| | | | dddddMMMMM | GTGAGCAG | |
| 262 | 262 | 5-10-5 | MMMMMddddd | TTTGGGCTGCTC | 167→186 |
| | | | dddddMMMMM | TATCTCCA | |
| 263 | 263 | 5-10-5 | MMMMMddddd | TTGTAGGGTTAA | 122→141 |
| | | | dddddMMMMM | TATGCAGA | |
| 264 | 264 | 5-10-5 | MMMMMddddd | AGTAGACATATT | 232→251 |
| | | | dddddMMMMM | CTCAGCTC | |
| 265 | 265 | 5-10-5 | MMMMMddddd | GGCAGGCCTCTG | 328→347 |
| | | | dddddMMMMM | GAATGAAG | |
| 266 | 266 | 5-10-5 | MMMMMddddd | TTCTATGTGAGC | 371→390 |
| | | | dddddMMMMM | AGCTCTTC | |
| 267 | 267 | 5-10-5 | MMMMMddddd | GAGGAGCCATTT | 139→158 |
| | | | dddddMMMMM | CCAACTTG | |
| 268 | 268 | 5-10-5 | MMMMMddddd | TCAGAGAGTTTT | 211→230 |
| | | | dddddMMMMM | GCATTATG | |
| 269 | 269 | 5-10-5 | MMMMMddddd | TCCTCAGAGAGT | 214→233 |
| | | | dddddMMMMM | TTTGCATT | |
| 270 | 270 | 5-10-5 | MMMMMddddd | TATCTCCAGTGT | 155→174 |
| | | | dddddMMMMM | TTGAGAGG | |
| 271 | 271 | 5-10-5 | MMMMMddddd | CCAACTTGTAGG | 127→146 |
| | | | dddddMMMMM | GTTAATAT | |
| 272 | 272 | 5-10-5 | MMMMMddddd | GATACATTTGGG | 173→192 |
| | | | dddddMMMMM | CTGCTCTA | |
| 273 | 273 | 5-10-5 | MMMMMddddd | TTGTCATTGTTA | 19→38 |
| | | | dddddMMMMM | TTATGGGT | |
| 274 | 274 | 5-10-5 | MMMMMddddd | TATGAAAGTGTT | 29→48 |
| | | | dddddMMMMM | GTCATTGT | |
| 275 | 275 | 5-10-5 | MMMMMddddd | TGATCTAAACTG | 408→427 |
| | | | dddddMMMMM | GTGAAAAT | |
| 276 | 276 | 5-10-5 | MMMMMddddd | CATATTCTCAGC | 226→245 |
| | | | dddddMMMMM | TCCTCAGA | |
| 277 | 277 | 5-10-5 | MMMMMddddd | AGAGGAGCCATT | 140→159 |
| | | | dddddMMMMM | TCCAACTT | |
| 278 | 278 | 5-10-5 | MMMMMddddd | GAAGGCACCAC | 191→210 |
| | | | dddddMMMMM | AGTAGCAGA | |
| 279 | 279 | 5-10-5 | MMMMMddddd | CATTTGGGCTGC | 169→188 |
| | | | dddddMMMMM | TCTATCTC | |
| 280 | 280 | 5-10-5 | MMMMMddddd | GAAAATGGAAG | 394→413 |
| | | | dddddMMMMM | TCACTACAA | |
| 281 | 281 | 5-10-5 | MMMMMddddd | CTCTCTGTCCAC | 420→439 |
| | | | dddddMMMMM | TGATCTAA | |
| 282 | 282 | 5-10-5 | MMMMMddddd | AGAGAGTTTTGC | 209→228 |
| | | | dddddMMMMM | ATTATGGA | |
| 283 | 283 | 5-10-5 | MMMMMddddd | TCCACTGATCTA | 413→432 |
| | | | dddddMMMMM | AACTGGTG | |
| 284 | 284 | 5-10-5 | MMMMMddddd | GCCATTTCCAAC | 134→153 |
| | | | dddddMMMMM | TTGTAGGG | |
| 285 | 285 | 5-10-5 | MMMMMddddd | GTTTGAGAGGAG | 145→164 |
| | | | dddddMMMMM | CCATTTCC | |
| 286 | 286 | 5-10-5 | MMMMMddddd | GAGCAGCTCTTC | 363→382 |
| | | | dddddMMMMM | AGCCTATA | |
| 287 | 287 | 5-10-5 | MMMMMddddd | CTTGTAGGGTTA | 123→142 |
| | | | dddddMMMMM | ATATGCAG | |
| 288 | 288 | 5-10-5 | MMMMMddddd | TTGAGAGGAGCC | 143→162 |
| | | | dddddMMMMM | ATTTCCAA | |
| 289 | 289 | 5-10-5 | MMMMMddddd | CTCCTCAGAGAG | 215→234 |
| | | | dddddMMMMM | TTTTGCAT | |
| 290 | 290 | 5-10-5 | MMMMMddddd | TAGCAGTAGACA | 236→255 |
| | | | dddddMMMMM | TATTCTCA | |
| 291 | 291 | 5-10-5 | MMMMMddddd | GAGTTTTGCATT | 206→225 |
| | | | dddddMMMMM | ATGGAAGG | |
| 292 | 292 | 5-10-5 | MMMMMddddd | AACTTGTAGGGT | 125→144 |
| | | | dddddMMMMM | TAATATGC | |
| 293 | 293 | 5-10-5 | MMMMMddddd | GTCCACTGATCT | 414→433 |
| | | | dddddMMMMM | AAACTGGT | |
| 294 | 294 | 5-10-5 | MMMMMddddd | GTAGGGTTAATA | 120→139 |
| | | | dddddMMMMM | TGCAGACT | |
| 295 | 295 | 5-10-5 | MMMMMddddd | AGCTCTTCAGCC | 359→378 |
| | | | dddddMMMMM | TATAGTGA | |
| 296 | 296 | 3-10-3 | CCCdddddddddd | TCTTGAGCAGTA | 445→460 |
| | | | CCC | ATTC | |
| 297 | 297 | 3-10-3 | CCCdddddddddd | CAGACTATACAG | 522→537 |
| | | | CCC | TTTA | |

As UMLILO is a lnRNA that regulates IL-8 transcription, the compounds were analyzed for their effect on IL-8 transcription by quantitative real-time PCR. The compounds were analyzed for their effect on cytotoxicity by assaying TNFRSF10b transcription by quantitative real-time PCR. The compounds were also analyzed for their effect on Toll-like receptor (TLR) signaling activation by assaying for transcription of the secreted embryonic alkaline phosphatase (SEAP) reporter gene transcription by quantitative real-time PCR. Data are averages from three experiments in which THP1 cells were treated with the antisense oligonucleotides of Table 1. If present, “N.D.” indicates “no data”. Data is represented as fold change relative to the RPL37A housekeeping gene.

TABLE 2
Inhibition of IL-8 transcription, TNFRSF10B expression and SEAP
expression in THP1 cells in the presence of gapmer compounds
of the present disclosure. The measured expression of IL-8, TNFRSF10B,
and SEAP is provided relative to the expression of
the housekeeping gene RPL37A. An expression value <1.0
means that the transcription of that gene was inhibited. For example,
a value of 0.25 means that gene transcription was inhibited by 75%.

| GAPMER | | | | |
|---|---|---|---|---|
| COM- | SEQ | | | |
| POUND | ID | IL-8 | TNFRSF10B | SEAP |
| NO. | NO: | EXPRESSION | EXPRESSION | EXPRESSION |
| 1 | 1 | 1.096 | 4.667 | 0.998 |
| 2 | 2 | 0.960 | 3.223 | 1.010 |
| 3 | 3 | 1.193 | 3.664 | 0.966 |
| 4 | 4 | 0.924 | 1.830 | 0.773 |
| 5 | 5 | 1.318 | 4.000 | 1.123 |
| 6 | 6 | 0.774 | 2.635 | 0.794 |
| 7 | 7 | 1.282 | 3.848 | 0.901 |
| 8 | 8 | 1.058 | 3.373 | 0.993 |
| 9 | 9 | 0.688 | 1.013 | 0.846 |
| 10 | 10 | 0.744 | 0.452 | 1.114 |
| 11 | 11 | 0.572 | 0.576 | 0.835 |
| 12 | 12 | 0.254 | 0.212 | 0.807 |
| 13 | 13 | 1.460 | 2.433 | 1.261 |
| 14 | 14 | 0.928 | 2.501 | 0.898 |
| 15 | 15 | 0.671 | 1.400 | 0.818 |
| 16 | 16 | 0.761 | 1.879 | 0.823 |
| 17 | 17 | 1.194 | 3.573 | 0.887 |
| 18 | 18 | 0.812 | 2.194 | 0.676 |
| 19 | 19 | 0.870 | 1.332 | 0.694 |
| 20 | 20 | 0.805 | 0.959 | 0.823 |
| 21 | 21 | 0.309 | 1.725 | 0.716 |
| 22 | 22 | 0.530 | 0.829 | 0.509 |
| 23 | 23 | 0.641 | 1.087 | 0.866 |
| 24 | 24 | 0.936 | 2.424 | 0.829 |
| 25 | 25 | 0.858 | 1.771 | 1.073 |
| 26 | 26 | 0.662 | 1.815 | 0.843 |
| 27 | 27 | 0.542 | 1.482 | 0.735 |
| 28 | 28 | 0.722 | 1.704 | 0.778 |
| 29 | 29 | 0.998 | 3.258 | 1.160 |
| 30 | 30 | 0.980 | 2.000 | 0.840 |
| 31 | 31 | 0.647 | 2.055 | 0.780 |
| 32 | 32 | 0.584 | 1.456 | 0.490 |
| 33 | 33 | 0.775 | 1.634 | 0.846 |
| 34 | 34 | 0.419 | 0.700 | 0.553 |
| 35 | 35 | 0.273 | 0.761 | 0.522 |
| 36 | 36 | 0.457 | 1.104 | 0.627 |
| 37 | 37 | 0.278 | 0.462 | 0.431 |
| 38 | 38 | 0.617 | 1.199 | 1.230 |
| 39 | 39 | 0.501 | 0.692 | 0.667 |
| 40 | 40 | 0.663 | 1.263 | 0.733 |
| 41 | 41 | 0.574 | 1.433 | 0.702 |
| 42 | 42 | 0.642 | 1.046 | 0.722 |
| 43 | 43 | 0.881 | 0.777 | 0.883 |
| 44 | 44 | 1.297 | 2.451 | 1.807 |
| 45 | 45 | 0.775 | 1.131 | 1.061 |
| 46 | 46 | 2.899 | 3.167 | 2.486 |
| 47 | 47 | 2.192 | 2.358 | 2.413 |
| 48 | 48 | 2.164 | 2.185 | 2.769 |
| 49 | 49 | 2.405 | 2.506 | 2.385 |
| 50 | 50 | 2.158 | 1.862 | 2.697 |
| 51 | 51 | 2.031 | 1.130 | 2.483 |
| 52 | 52 | 2.279 | 1.105 | 2.021 |
| 53 | 53 | 1.307 | 0.606 | 2.154 |
| 54 | 54 | 1.568 | 0.058 | 2.121 |
| 55 | 55 | 0.747 | 0.112 | 0.023 |
| 56 | 56 | 0.397 | 0.082 | 1.234 |
| 57 | 57 | 1.342 | 1.783 | 1.684 |
| 58 | 58 | 1.620 | 1.841 | 1.932 |
| 59 | 59 | 1.647 | 2.038 | 2.417 |
| 60 | 60 | 2.273 | 1.671 | 2.526 |
| 61 | 61 | 1.319 | 2.015 | 1.252 |
| 62 | 62 | 0.900 | 1.480 | 1.944 |
| 63 | 63 | 1.251 | 0.876 | 1.236 |
| 64 | 64 | 1.042 | 1.997 | 1.573 |
| 65 | 65 | 0.620 | 1.519 | 0.980 |
| 66 | 66 | 0.425 | 0.215 | 0.639 |
| 67 | 67 | 0.855 | 2.265 | 0.981 |
| 68 | 68 | 1.246 | 1.851 | 0.932 |
| 69 | 69 | 1.153 | 1.799 | 1.247 |
| 70 | 70 | 1.033 | 1.803 | 0.912 |
| 71 | 71 | 1.024 | 3.442 | 1.202 |
| 72 | 72 | 1.491 | 4.053 | 1.417 |
| 73 | 73 | 1.574 | 3.240 | 1.747 |
| 74 | 74 | 1.927 | 1.395 | 1.376 |
| 75 | 75 | 2.805 | 4.831 | 1.768 |
| 76 | 76 | 0.668 | 0.266 | 0.898 |
| 77 | 77 | 0.546 | 0.342 | 0.711 |
| 78 | 78 | 0.571 | 0.244 | 0.695 |
| 79 | 79 | 0.548 | 0.325 | 0.755 |
| 80 | 80 | 0.867 | 0.256 | 0.760 |
| 81 | 81 | 0.744 | 0.241 | 0.769 |
| 82 | 82 | 1.837 | 0.951 | 1.623 |
| 83 | 83 | 1.557 | 0.981 | 1.387 |
| 84 | 84 | 2.183 | 0.743 | 1.331 |
| 85 | 85 | 2.603 | 0.863 | 2.639 |
| 86 | 86 | 1.654 | 1.366 | 1.221 |
| 87 | 87 | 1.204 | 0.910 | 0.925 |
| 88 | 88 | 0.259 | 0.742 | 0.723 |
| 89 | 89 | 0.836 | 3.897 | 1.115 |
| 90 | 90 | 1.026 | 3.501 | 0.970 |
| 91 | 91 | 1.411 | 2.917 | 1.801 |
| 92 | 92 | 1.426 | 3.228 | 1.552 |
| 93 | 93 | 2.774 | 2.521 | 1.523 |
| 94 | 94 | 1.605 | 2.031 | 1.122 |
| 95 | 95 | 0.574 | 1.500 | 0.528 |
| 96 | 96 | 1.519 | 2.722 | 0.892 |
| 97 | 97 | 2.084 | 3.286 | 0.994 |
| 98 | 98 | 1.704 | 2.208 | 1.078 |
| 99 | 99 | 2.146 | 2.916 | 0.692 |
| 100 | 100 | 0.155 | 0.759 | 0.759 |
| 101 | 101 | 0.360 | 0.304 | 0.711 |
| 102 | 102 | 0.237 | 0.279 | 0.531 |
| 103 | 103 | 0.511 | 0.377 | 0.721 |
| 104 | 104 | 0.821 | 0.351 | 0.920 |
| 105 | 105 | 0.962 | 0.586 | 0.837 |
| 106 | 106 | 1.119 | 0.456 | 1.047 |
| 107 | 107 | 19.302 | 3.904 | 4.880 |
| 108 | 108 | 2.343 | 4.016 | 2.870 |
| 109 | 109 | 1.911 | 5.919 | 3.429 |
| 110 | 110 | 1.533 | 2.704 | 2.574 |
| 111 | 111 | 54.319 | 6.903 | 7.427 |
| 112 | 112 | 1.977 | 3.893 | 3.455 |
| 113 | 113 | 4.396 | 3.231 | 3.028 |
| 114 | 114 | 2.748 | 3.386 | 3.441 |
| 115 | 115 | 4.002 | 4.349 | 3.218 |
| 116 | 116 | 2.004 | 3.750 | 2.898 |
| 117 | 117 | 11.948 | 4.504 | 4.154 |
| 118 | 118 | 3.752 | 3.619 | 3.504 |
| 119 | 119 | 12.846 | 2.109 | 4.734 |
| 120 | 120 | 7.495 | 2.331 | 0.003 |
| 121 | 121 | 1.103 | 0.732 | 1.698 |
| 122 | 122 | 0.975 | 0.328 | 2.217 |
| 123 | 123 | 0.160 | 0.091 | 2.194 |
| 124 | 124 | 0.275 | 0.128 | 2.011 |
| 125 | 125 | 11.103 | 0.448 | 4.928 |
| 126 | 126 | 0.973 | 0.300 | 2.248 |
| 127 | 127 | 0.249 | 0.084 | 1.622 |
| 128 | 128 | 0.536 | 0.150 | 1.632 |
| 129 | 129 | 4.980 | 0.206 | 3.922 |
| 130 | 130 | 12.764 | 1.727 | 1.887 |
| 131 | 131 | 4.890 | 4.117 | 4.790 |
| 132 | 132 | 12.554 | 4.448 | 2.772 |
| 133 | 133 | 7.577 | 4.847 | 3.115 |
| 134 | 134 | 9.360 | 5.616 | 4.396 |
| 135 | 135 | 62.253 | 6.826 | 5.210 |
| 136 | 136 | 4.120 | 3.876 | 2.499 |
| 137 | 137 | 57.859 | 6.329 | 4.626 |
| 138 | 138 | 50.641 | 6.951 | 7.914 |
| 139 | 139 | 2.826 | 3.439 | 3.260 |
| 140 | 140 | 3.046 | 1.943 | 2.122 |
| 141 | 141 | 22.025 | 4.840 | 5.183 |
| 142 | 142 | 1.670 | 1.997 | 2.066 |
| 143 | 143 | 2.711 | 0.928 | 3.222 |
| 144 | 144 | 2.697 | 1.340 | 2.762 |
| 145 | 145 | 1.683 | 0.916 | 2.285 |
| 146 | 146 | 7.114 | 2.051 | 3.206 |
| 147 | 147 | 2.407 | 0.894 | 2.012 |
| 148 | 148 | 1.740 | 0.694 | 2.335 |
| 149 | 149 | 1.183 | 0.560 | 1.363 |
| 150 | 150 | 0.404 | 0.457 | 0.530 |
| 233 | 233 | 1.326 | 2.351 | 1.340 |
| 234 | 234 | 1.235 | 3.199 | 1.954 |
| 235 | 235 | 1.558 | 3.971 | 2.033 |
| 236 | 236 | 1.254 | 3.245 | 1.811 |
| 237 | 237 | 1.525 | 3.456 | 1.667 |
| 238 | 238 | 2.235 | 2.790 | 1.861 |
| 239 | 239 | 2.823 | 3.031 | 1.986 |
| 240 | 240 | 2.360 | 3.004 | 2.240 |
| 241 | 241 | 1.988 | 3.508 | 2.023 |
| 242 | 242 | 2.411 | 3.263 | 2.289 |
| 243 | 243 | 1.917 | 2.389 | 1.622 |
| 244 | 24 | 1.623 | 2.043 | 1.200 |
| 245 | 245 | 1.475 | 1.185 | 1.214 |
| 246 | 246 | 2.614 | 2.512 | 1.343 |
| 247 | 247 | 2.766 | 2.084 | 1.376 |
| 248 | 248 | 2.997 | 1.924 | 1.242 |
| 249 | 249 | 1.932 | 1.495 | 1.081 |
| 250 | 250 | 2.472 | 2.178 | 1.306 |
| 251 | 251 | 2.450 | 2.890 | 1.475 |
| 252 | 252 | 2.814 | 2.371 | 1.755 |
| 253 | 253 | 1.841 | 2.574 | 1.396 |
| 254 | 254 | 2.237 | 1.550 | 1.327 |
| 255 | 255 | 2.646 | 3.699 | 1.621 |
| 256 | 256 | 2.631 | 2.649 | 2.041 |
| 257 | 257 | 2.669 | 2.475 | 1.716 |
| 258 | 258 | 2.637 | 3.208 | 2.162 |
| 259 | 259 | 2.216 | 2.062 | 1.210 |
| 260 | 260 | 1.804 | 2.333 | 1.869 |
| 261 | 261 | 1.265 | 0.916 | 1.083 |
| 262 | 262 | 1.242 | 0.737 | 1.617 |
| 263 | 263 | 1.075 | 0.519 | 1.071 |
| 264 | 264 | 0.930 | 0.748 | 1.196 |
| 265 | 265 | 0.903 | 0.416 | 1.318 |
| 266 | 266 | 1.090 | 0.227 | 1.707 |
| 267 | 267 | 3.119 | 2.542 | 1.554 |
| 268 | 268 | 4.003 | 2.729 | 1.941 |
| 269 | 269 | 2.443 | 2.244 | 1.950 |
| 270 | 270 | 3.151 | 2.004 | 1.906 |
| 271 | 271 | 1.987 | 2.264 | 1.662 |
| 272 | 272 | 2.184 | 2.471 | 1.459 |
| 273 | 273 | 1.072 | 0.896 | 0.880 |
| 274 | 274 | 1.070 | 0.999 | 1.045 |
| 275 | 275 | 0.785 | 0.936 | 0.845 |
| 276 | 276 | 0.914 | 0.879 | 1.204 |
| 277 | 277 | 0.841 | 0.820 | 0.797 |
| 278 | 278 | 0.860 | 0.808 | 0.861 |
| 279 | 279 | 1.070 | 1.102 | 0.976 |
| 280 | 280 | 0.729 | 0.488 | 0.528 |
| 281 | 281 | 0.787 | 0.543 | 0.629 |
| 282 | 282 | 0.945 | 0.767 | 0.967 |
| 283 | 283 | 0.958 | 1.382 | 1.394 |
| 284 | 284 | 1.205 | 1.693 | 1.911 |
| 285 | 285 | 0.913 | 1.245 | 1.410 |
| 286 | 286 | 1.276 | 1.585 | 1.821 |
| 287 | 287 | 1.074 | 1.467 | 1.674 |
| 288 | 288 | 1.059 | 1.420 | 1.849 |
| 289 | 289 | 0.807 | 0.950 | 0.861 |
| 290 | 290 | 1.061 | 1.240 | 1.408 |
| 291 | 291 | 0.929 | 0.934 | 1.267 |
| 292 | 292 | 1.182 | 1.285 | 1.473 |
| 293 | 293 | 0.922 | 1.251 | 1.251 |
| 294 | 294 | 0.728 | 1.296 | 1.212 |
| 295 | 295 | 1.044 | 1.239 | 1.105 |

Gapmer compounds SEQ TD NOs: 12, 21, 35, 37, 88, 100, 102, 123, 124, and 127 demonstrated at least 70% inhibition of human IL-8 expression in this assay. As further shown in Table 2, gapmer compounds SEQ TD NOs: 12, 35, 37, 88, 100, 102, 123, 124, and 127 demonstrated zero or up to 5000 inhibition of TNFRSF10b (a measure of cytotoxicity), which is low cytotoxicity. SEQ TD NOs: 12, 21, 35, 37, 88, 100, 102, and 127 demonstrated zero or up to 50% inhibition of SEAP (a measure of immune activation), indicating low immune stimulatory activity.

Table 3 shows inhibition of IL-8 expression by chimeric phosphorothioate gapmers SEQ ID NOs 152-222 that target UMLILO (SEQ ID NO: 231). Data is represented as fold change relative to the RPL37A housekeeping gene.

TABLE 3
Inhibition results of UMLILO and corresponding gene inhibition. A
value less than 1, represents inhibition.

| GAPMER | | | | |
|---|---|---|---|---|
| COM- | SEQ | | | |
| POUND | ID | IL-8 | TNFRSF10B | SEAP |
| NO. | NO: | EXPRESSION | EXPRESSION | EXPRESSION |
| 152 | 152 | 0.841 | 0.918 | 1.359 |
| 153 | 153 | 0.909 | 1.253 | 1.253 |
| 155 | 155 | 0.802 | 1.244 | 1.473 |
| 156 | 156 | 0.483 | 0.78 | 1.283 |
| 157 | 157 | 0.611 | 0.871 | 1.413 |
| 158 | 158 | 0.369 | 0.7 | 1.264 |
| 159 | 159 | 0.403 | 0.575 | 1.259 |
| 160 | 160 | 0.35 | 0.648 | 1.148 |
| 161 | 161 | 0.302 | 0.705 | 1.374 |
| 162 | 162 | 0.557 | 0.626 | 1.045 |
| 164 | 164 | 0.876 | 1.173 | 1.348 |
| 165 | 165 | 0.632 | 0.95 | 1.04 |
| 166 | 166 | 0.422 | 0.718 | 0.979 |
| 167 | 167 | 0.513 | 0.967 | 0.935 |
| 168 | 168 | 0.307 | 0.495 | 0.661 |
| 169 | 169 | 0.274 | 0.764 | 1.012 |
| 170 | 170 | 0.387 | 0.705 | 1.254 |
| 171 | 171 | 0.321 | 0.176 | 0.071 |
| 172 | 172 | 0.389 | 1.09 | 1.422 |
| 173 | 173 | 0.218 | 0.503 | 0.237 |
| 174 | 174 | 0.948 | 1.629 | 1.106 |
| 176 | 176 | 1.472 | 1.106 | 1.09 |
| 177 | 177 | 1.155 | 0.875 | 1.227 |
| 178 | 178 | 1.213 | 1.094 | 1.32 |
| 179 | 179 | 0.909 | 1.032 | 1.363 |
| 180 | 180 | 0.687 | 1.233 | 1.227 |
| 181 | 181 | 1.162 | 1.059 | 1.105 |
| 182 | 182 | 1.148 | 1.382 | 0.983 |
| 183 | 183 | 1.086 | 1.306 | 1.157 |
| 184 | 184 | 1.099 | 1.715 | 1.169 |
| 185 | 185 | 1.107 | 1.025 | 1.157 |
| 186 | 186 | 1.22 | 1.37 | 1.139 |
| 188 | 188 | 0.445 | 0.833 | 0.793 |
| 189 | 189 | 0.814 | 0.828 | 0.792 |
| 190 | 190 | 0.617 | 0.724 | 0.794 |
| 191 | 191 | 0.656 | 0.872 | 0.883 |
| 192 | 192 | 0.553 | 0.729 | 0.743 |
| 193 | 193 | 0.716 | 0.745 | 0.723 |
| 194 | 194 | 0.595 | 0.85 | 0.756 |
| 195 | 195 | 0.689 | 0.753 | 0.619 |
| 196 | 196 | 0.469 | 0.773 | 0.513 |
| 197 | 197 | 0.31 | 1.011 | 0.6 |
| 198 | 198 | 0.258 | 0.815 | 0.476 |
| 199 | 199 | 0.923 | 0.984 | 0.828 |
| 200 | 200 | 0.679 | 0.947 | 1.064 |
| 201 | 201 | 1.117 | 1.391 | 1.394 |
| 202 | 202 | 0.778 | 0.856 | 0.92 |
| 203 | 203 | 0.709 | 0.905 | 1.316 |
| 204 | 204 | 1.299 | 1.484 | 1.621 |
| 205 | 205 | 1.18 | 1.55 | 1.895 |
| 206 | 206 | 0.943 | 1.349 | 1.384 |
| 207 | 207 | 0.96 | 1.447 | 0.735 |
| 209 | 209 | 0.839 | 0.198 | 0.236 |
| 210 | 210 | 1.302 | 1.158 | 0.978 |
| 211 | 211 | 1.098 | 1.209 | 1.037 |
| 212 | 212 | 0.77 | 1.297 | 0.7 |
| 213 | 213 | 0.916 | 0.921 | 0.595 |
| 214 | 214 | 0.769 | 1.098 | 0.668 |
| 215 | 215 | 0.769 | 1.044 | 0.721 |
| 216 | 216 | 0.467 | 1.212 | 0.551 |
| 217 | 217 | 0.711 | 1.629 | 1.066 |
| 218 | 218 | 1.105 | 1.514 | 1.196 |
| 219 | 219 | 1.64 | 1.745 | 1.264 |
| 221 | 221 | 1.115 | 1.219 | 1.049 |
| 222 | 222 | 0.93 | 1.173 | 2.27 |
| 233 | 233 | 0.467 | 0.771 | 1.121 |
| 296 | 296 | 0.51 | N.D. | N.D. |
| 297 | 297 | 0.55 | N.D. | N.D. |

Based on the screening data in Tables 1-4, six regions on the target UMLILO sequence (SEQ TD NO: 231) were found for gapmers SEQ ID NOs: 12; 21; 35; 37; 100; and 128. Tables 4A and 4B provide the average inhibition of (1) IL-8, (2) SLAP and (3) TNFRSF10b of the gapmers targeted to Regions A-F of UMLILO.

TABLE 4A

| | | SEQ | Gapmer position on | UMLILO Target |
|---|---|---|---|---|
| UMLILO | GAPMER | ID | UMLILO | Region SEQ |
| Region ID | NO. | NO: | sequence 231 | ID NO: 231 |
| A | 12 | 12 | 263-282 | 256-285 |
| B | 21 | 21 | 520-539 | 511-540 |
| C | 35 | 35 | 527-546 | 523-547 |
| D | 37 | 37 | 442-460 | 441-469 |
| E | 100 | 100 | 91-106 | 88-107 |
| F | 128 | 128 | 547-562 | 547-567 |

TABLE 4B

| UMLILO | | SEQ | Average | Average | Average |
|---|---|---|---|---|---|
| Region | GAPMER | ID | IL-8 | SEAP % | TNFRSF10b |
| ID | NO. | NO: | % inhibition | inhibition | % inhibition |
| A | 12 | 12 | 70.5 | 36.8 | 40.7 |
| B | 21 | 21 | 82.6 | 45.8 | 31.8 |
| C | 35 | 35 | 77.6 | 42.8 | 24.1 |
| D | 37 | 37 | 71.9 | 31.1 | 50.7 |
| E | 100 | 100 | 77.1 | 31.2 | 39.9 |
| F | 128 | 128 | 73.6 | 21.2 | 13.9 |

All of the gapmers targeted to Regions A-F of UMLILO, at positions of: 256-285, 511-540, 523-547, 441-469, 88-107 and 547-567, respectively, each demonstrated more than 70% inhibition of TL-8 expression. Furthermore, there was more than 20% reduction in SEAP activity for the gapmers tested in Table 4A & 4B. Region D (positions 441-469 of UMLILO SEQ ID NO: 231) demonstrated the lowest overall cytotoxicity. Gapmers targeting Region D are selected from the group consisting of SEQ ID NOs: 36, 37, 38, 39, 40, 41, 42, 55, 56, 152, 153, 155, 156, 157, 158, 159, 160, 161, 162, 223, and 224.

Designing and Testing Different Species UMLILO Cross-Reacting Antisense Compounds:

Human and porcine UMLILO target sequences were compared for regions of homology but none were found to be as long as 20 nucleotides. However, based on the sequence homology between the human and porcine UMLILO target sequences, a series of gapmer antisense sequences were designed which were complementary to either human and porcine UMLILO and which had no more than 1 mismatch to human and porcine UMLILO.

Thus, such gapmers were designed to work in both in vitro models with human cells and in porcine in vivo models. However, the relative antisense efficacy may not be equal for the two forms because of imperfect homology to one UMLILO or the other.

Table 5 shows the sequence of 5 more active gapmers as a third group of screened gapmers. SEQ ID NO: 223, 225, 227 are 100% complimentary to human UMLILO. SEQ ID NO: 224 and 226 have a single mismatch to human UMLILO and are 100% complimentary to porcine UMLILO (SEQ ID NO: 232); (5′ GTTACATGTAGAGATGGAAACTTGCAATAACAATGGATCAAACCCTCACAATGCTA GCTGTCACCATATTAGGCTAGATGATAGAAACATGTGAATAACTGCTCAAGAAAAT ATAGAACCACATCCTTTGAAATTCAGAAGCTTCAACTGGGAGGGCTCTTGAGCCTG CTGGACTGTATACTCTGTAAAAACAGAACTGTCTTCGTCTCACTCACTATTTTA 3′).

TABLE 5
Gapmer compound tested for binding to human and porcine UMLILO
Nucleoside modified chemistries: M = MOE; L = Locked Nucleic Acid (cMe modified
nucleoside); 2′M = 2′OMe; d = 2′deoxynucleotide.

| Gapmer | SEQ | | | Sequence of | Complementary |
|---|---|---|---|---|---|
| Compound | ID | | | gapmer | to human |
| No. | NO: | Configuration | Modification | compound | UMLILO position |
| 223 | 223 | 4-10-4 | MMMMddddd | TTCTTGAGCA | 444→461 |
| | | | dddddMMMM | GTAATTCA | |
| 224 | 224 | 4-10-4 | MMMMddddd | TTCTTGAGCA | 444→461 |
| | | | dddddMMMM | GTTATTCA | |
| 225 | 225 | 3-10-3 | LLLdddddddd | CTTGAGCAGT | 444→459 |
| | | | ddLLL | AATTCA | |
| 226 | 226 | 3-10-3 | LLLdddddddd | CTTGAGCAGT | 444→459 |
| | | | ddLLL | TATTCA | |
| 227 | 227 | 3-10-3 | LLLdd2′Mddd | CTTGAGCAGT | 444→459 |
| | | | ddddLLL | AATTCA | |

### Example 2. In Vitro Inhibition of UMLILO Transcription

This example shows the effect of UMLILO inhibition in THP1s with the candidate gapmer compounds determined by UMLILO mRNA expression in gapmer compound treated THP1s by quantitative real-time PCR. Gapmers were tested as percent inhibition of UMLILO expression relative to control gapmer (AACACGTCTATACGC SEQ ID 228). Each gapmer concentration was 10 μM and was incubated with cells for 48 hours. Data is represented in Table 6 as % inhibition of UMLILO relative to control gapmer treated cells.

TABLE 6

| GAPMER | | |
|---|---|---|
| COMPOUND NO. | SEQ ID NO: | % inhibition |
| 150 | 150 | 40 |
| 12 | 12 | 64 |
| 21 | 21 | 66 |
| 35 | 35 | 68 |
| 37 | 37 | 62 |
| 100 | 100 | 60 |
| 128 | 128 | 73 |
| | 228 | 0 |

Gapmer SEQ ID NO 12, 21, 35, 37, 100 and 128 demonstrated at least 60% inhibition of human UMLILO expression in the THP1s and are superior to gapmer SEQ ID NO 150.

### Example 3. In Vitro UMLILO Expression Inhibition in Human Primary Monocytes

This example shows UMLILO expression in primary human monocytes with candidate gapmer compounds determined by UMLILO mRNA expression in gapmer compound treated human primary monocytes by quantitative real-time PCR. Two gapmer compounds were tested to measure percent inhibition of UMLILO present in human primary monocytes. The results obtained are expressed as percent inhibition of UMLILO expression relative to negative control, a gapmer compound control that is not complementary to any UMLILO sequence (AACACGTCTATACGC SEQ ID 228). Each gapmer compound concentration was 10 μM. SEQ ID NO: 223 is 100% complimentary to bases 444 to 461 of human UMLILO (SEQ ID NO: 231).

TABLE 7

| GAPMER | SEQ | | | |
|---|---|---|---|---|
| COMPOUND NO. | ID NO: | Donor 1 | Donor 2 | Donor 3 |
| 223 | 223 | 95 | 66 | 80 |
| 150 | 150 | 92 | N.D. | 40 |
| 228 | 228 | 0 | 0 | 0 |

Gapmer SEQ ID NO 223 demonstrated at least 66% inhibition of human UMLILO expression in the monocytes from three separate donors (Table 7).

### Example 4. Inhibition of IL-8 Expression in PBMCs Via UMLILO Inhibition with Gapmer Compounds

This example provides the results of an experiment to determine the effect of UMLILO inhibition on cytokine protein level production and expression in unstimulated PBMCs. Peripheral blood mononuclear cells (PBMC) were isolated from individuals and separated from other components of blood (such as erythrocytes and granulocytes), via density gradient centrifugation using Ficoll-Pague (GE Healthcare). PBMCs were maintained in RPMI 1640 media. Gapmer compounds were delivered into cells by gymnosis (See for example, methods described in Soifer, H. et al., (2012) “Silencing of gene expression by gymnotic delivery of antisense oligonucleotides” Methods Mol Biol., Vol. 815:333-46, the disclosure of which is incorporated herein by reference in its entirety). Gymnosis is a process for delivery of antisense oligodeoxynucleotides (such as gapmer compounds of the present disclosure) to cells, in the absence of any carriers or conjugation that produces sequence-specific gene silencing. TL-8 protein expression from treated PBMCs with the gapmer compounds was determined by ELISA. Data is represented as μg/mL of IL-8 protein. SEQ ID NO: 224 has a single mismatch to human UMLILO at base 449 of human UMLILO (SEQ ID NO: 231) and is 100% complimentary to porcine UMLILO (SEQ ID NO: 232).

TABLE 8
Inhibition of IL-8 expression in human PBMCs
when treated with gapmer compounds.

| | IL-8 expression (pg/mL) after exposure | | | | | |
|---|---|---|---|---|---|---|
| | to Gapmer compounds in human PBMCs | | | | | |
| SEQ ID NO: | Donor 1 | | | Donor 2 | | |
| (Gapmer | Gapmer compound concentration | | | | | |
| Compound No.) | 1 μM | 5 μM | 10 μM | 1 μM | 5 μM | 10 μM |
| 224 (224) | 43.00 | 5.99 | 14.22 | 53.51 | N.D. | 8.12 |
| 223 (223) | 307.96 | 126.28 | 14.22 | 79.91 | 19.63 | 14.11 |

Gapmer compounds SEQ ID NO 224 and 223 (Gapmer compounds 224 and 223) inhibited IL8 protein secretion in a dose-dependent manner in unstimulated PBMCs (Table 8).

### Example 5. UMLILO Inhibition on Cytokine Protein Levels in LPS-Stimulated PBMCs

This example shows an effect of UMLILO inhibition on cytokine protein levels in LPS-stimulated PBMCs. PBMCs were isolated from the individuals as in Example 3 and then stimulated with LPS (10 ng/mL; Sigma) for 24 hr to induce the expression of cytokines such as IL-8. Gapmer compounds (SEQ ID NO: 223 and 224) were delivered into cells by gymnosis as in Example 3. IL-8 protein expression was determined by ELISA. Data is represented as μg/mL of IL-8 protein expression. The results obtained are expressed as percent inhibition of IL-8 expression relative to negative control, a gapmer compound control that is not complementary to any UMLILO sequence (AACACGTCTATACGC SEQ ID 228).

TABLE 9
Secretion and expression of IL-8 (pg/mL) from LPS stimulated
PBMCs treated with gapmer compounds (SEQ ID Nos: 223 & 224)

| GAPMER | | | | | | | |
|---|---|---|---|---|---|---|---|
| COMPOUND | SEQ ID | Donor 1 | | | Donor 2 | | |
| NO. | NO: | 1 μM | 5 μM | 10 μM | 1 μM | 5 μM | 10 μM |
| 224 | 224 | 132.28 | 105.13 | 104.40 | 102.42 | 100.00 | 77.94 |
| 223 | 223 | 85.96 | 88.30 | 42.24 | 79.64 | 112.29 | 72.00 |

Gapmers SEQ TD NO 224 and 223 inhibited IL8 protein secretion in a dose-dependent manner in LPS-stimulated PBMCs. SEQ TD NO 223 demonstrated a higher potency for TL-8 inhibition relative to SEQ TD NO 224.

Table 10 shows Tumor Necrosis Factor (TNF) inhibition in cells treated with gapmer compounds. TNF protein expression was determined by ELISA. Data is represented as μg/mL of TNF protein.

TABLE 10
Levels of TNF secretion and expression (pg/mL) from LPS stimulated
PBMCs treated with gapmer compounds (SEQ ID NOs: 223 & 224).

| | | TNF expression (pg/mL) after exposure | | | | | |
|---|---|---|---|---|---|---|---|
| GAPMER | | to Gapmer compounds in human PBMCs | | | | | |
| COMPOUND | SEQ ID | Donor 1 | | | Donor 2 | | |
| NO. | NO: | 1 μM | 5 μM | 10 μM | 1 μM | 5 μM | 10 μM |
| 224 | 224 | 250.36 | 152.27 | 53.72 | 108.61 | 82.36 | 100.00 |
| 223 | 223 | 138.04 | 70.72 | 46.73 | 61.26 | 129.20 | N.D. |

Gapmers SEQ TD NO 224 and 223 (Gapmer compounds 224 and 223) inhibited TNF protein secretion in a dose-dependent manner in LPS-stimulated PBMCs. SEQ ID NO 223 demonstrated higher potency relative to SEQ TD NO 224.

### Example 6. Effect of Gapmer Compounds on the Expression of UMLILO RNA in LPS-Treated PBMCs

This example shows the effect of UMLILO inhibition on cytokine mRNA levels in LPS-stimulated human PBMCs. UMLILO mRNA expression was determined in gapmer compound-treated human PBMCs. The gapmers were analyzed for their effect on UMLILO transcription by quantitative real-time PCR. Table 11 shows the measured expression of UMLILO relative to the expression of the housekeeping gene RPL37A. An expression value <1.0 means that the transcription of that gene was inhibited.

TABLE 11
Levels of UMLILO RNA expression from LPS stimulated PBMCs treated
with gapmer compounds 223 and 224 (SEQ ID NOs: 223 & 224).

| GAPMER | SEQ ID | | | | | | | | | |
|---|---|---|---|---|---|---|---|---|---|---|
| COMPOUND | NO: | Donor 1 | | | Donor 2 | | | Donor 3 | | |
| NO. | Conc. | 1 μM | 5 μM | 10 μM | 1 μM | 5 μM | 10 μM | 1 μM | 5 μM | 10 μM |
| 224 | 224 | 1.58 | 0.43 | 0.33 | 0.79 | 1.61 | 0.18 | 0.84 | 0.66 | N.D. |
| 223 | 223 | 2.15 | 1.52 | 1.09 | 0.67 | 0.40 | 0.58 | 0.52 | 0.72 | 0.13 |

Gapmer compounds 224 and 223 inhibited UMLILO RNA expression in a dose-dependent manner in LPS-stimulated PBMCs. Gapmer compound 223 (SEQ TD NO: 223) demonstrated higher potency relative to SEQ TD NO 224.

TL-8 mRNA expression was determined in gapmer treated human PBMCs. The gapmers were analyzed for their effect on TL-8 transcription by quantitative real-time PCR. The measured expression of IL-8 is provided relative to the expression of the housekeeping gene RPL37A. An expression value <1.0 means that the transcription of that gene was inhibited.

TABLE 12
Inhibition of IL-8 expression in human LPS-treated PBMCs.

| GAPMER | SEQ ID | | | | | | | | | |
|---|---|---|---|---|---|---|---|---|---|---|
| COMPOUND | NO: | Donor 1 | | | Donor 2 | | | Donor 3 | | |
| NO. | Conc. | 1 μM | 5 μM | 10 μM | 1 μM | 5 μM | 10 μM | 1 μM | 5 μM | 10 μM |
| 224 | 224 | 1.39 | 1.29 | 0.28 | 1.23 | 0.62 | 0.45 | 1.22 | 1.51 | 0.37 |
| 223 | 223 | 1.10 | 0.80 | 0.23 | 0.60 | 0.97 | 0.35 | 0.13 | 0.32 | 0.33 |

Gapmer compounds 224 and 223 (SEQ ID NOs: 224 and 223) inhibited IL-8 RNA expression in LPS-stimulated PBMCs. SEQ ID NO: 223 demonstrated higher potency relative to SEQ ID NO: 224.

### Example 7. UMLILO Inhibition on Cytokine mRNA Levels in LPS-Stimulated Porcine Macrophages

This example shows the effect of UMLILO inhibition on cytokine mRNA levels in LPS-stimulated porcine macrophages. This was determined by UMLILO mRNA expression in gapmer compound treated porcine primary macrophages by quantitative real-time PCR. Two gapmers compounds, 223, and 224 (SEQ ID NOs: 223 and 224), and a control (AACACGTCTATACGC SEQ ID NO: 228) were tested. Table 13 shows percent inhibition relative to control oligonucleotide SEQ ID NO: 228. SEQ ID NO: 224 has a single mismatch to human UMLILO at base 449 of human UMLILO (SEQ ID NO: 331) and is 100% complimentary to porcine UMLILO (SEQ ID NO: 332).

TABLE 13
Percent inhibition of UMLILO expression in porcine macrophages
when treated with gapmer compounds 223 and 224
(SEQ ID NOs: 223 and 224).

| GAPMER | | |
|---|---|---|
| COMPOUND NO. | SEQ ID NO: | % inhibition |
| 223 | 223 | 8 |
| 224 | 224 | 55 |
| 228 | 228 | 0 |

Gapmer SEQ ID NO 224 demonstrated greater inhibition of porcine UMLILO relative to SEQ ID NO 223. Gapmer compound SEQ ID NO: 224 has 100% complementary sequence identity to a region on porcine UMLILO (SEQ ID NO: 232). SEQ ID NO:223 gapmer compound has a single mismatch to porcine UMLILO sequence SEQ ID NO: 232.

### Example 8. Inhibition of UMLILO Expression and Cytokine Production in Cell Culture with Rheumatoid Arthritis (RA) Synovial Explants

This example measured gapmer compound inhibition of UMLILO expression in synovial explant tissue from patients with rheumatoid arthritis (RA). During joint replacement surgery, human RA synovial tissue was collected in RPMI media containing gentamycin. The synovial tissue was immediately processed in synovial biopsies using skin biopsy punches of 3 mm. Per donor, 3 biopsies per experimental group were used which were randomly divided over the treatment groups. Table 14 shows percent inhibition relative to an unrelated control gapmer (AACACGTCTATACGC SEQ ID 228). The gapmer concentrations were 1p M and 5 μM. The biopsies were cultured in 200 μl in a 96-wells plate for 24 hours. At the end of culture, RA synovial explants were collected and cytokine levels were determined using Luminex bead array technology. Table 14 shows the percentage inhibition of IL-8, IL-6, IL-1B and TNF in the supernatant after 24 hours of culture. Numbers are the results of 3 separate experiments from 3 donors.

F=2′F-ANA modified nucleoside; d=DNA base

TABLE 14
Results of inhibition of cytokine production in cell cultures containing
human RA synovial explants when incubated with a gapmer compound 230 (SEQ ID NO: 230).

| SEQ ID NO: (Gapmer | | Nucleoside | Gapmer Compound |
|---|---|---|---|
| Compound NO.) | Configuration | modification Chemistry | Sequence |
| 230 (230) | 3-10-3 | FFFddddddddddFFF | TCGCCTCTAATTT |
| | | | AAG |

| | % inhibition of cytokine (pg/mL) in cell culture after | | | | | | | |
|---|---|---|---|---|---|---|---|---|
| | incubation with RA synovial explants | | | | | | | |
| Treatment | IL-8 | | IL-6 | | IL-1B | | TNF | |
| Dose of gapmer | 1 μM | 5 μM | 1 μM | 5 μM | 1 μM | 5 μM | 1 μM | 5 μM |
| compound | | | | | | | | |
| (SEQ ID NO: 230; | 26.48 | 38.65 | 31.76 | 47.37 | 24.72 | 81.27 | 60.30 | 69.77 |
| GAPMER NO: 230) | | | | | | | | |

Gapmer compound 230 (SEQ ID NO: 230) reduced TL-8, IL-6, IL-1B and TNF cytokine levels secreted from the biopsies in a dose-dependent manner.

### Example 9. In Vivo Analysis of Gapmer Compound Activity in a Porcine Neovascularization Model

This example provides an in vivo study of gapmer compound administration directly to the eyes in pigs for induced angiogenic conditions in the eye in a pig model of choroidal neovascularization (CNV) to study ocular neovascularization. Male farm pigs (8-10 kg) were subjected to CNV lesions by laser treatment in both eyes. The extent of CNV was determined by fluorescein angiography after a 2 week period. Due to its higher potency demonstrated in porcine cells, a single intra-vitreous injection (7.8 μM or 15 μM) of gapmer compound 224 (SEQ ID NO: 224) in 50 μl saline was performed on the day of CNV induction. Five pigs were included in each of the three treatment groups (saline, 7.8 μM or 15 μM) and the intravitreal injection was performed in both eyes (n=10 eyes per group). Fluorescein angiography was performed at day 14 following intravitresl injections to measure the neovascular response. Measurements are represented as corrected total cell fluorescence (CTLF). Reduced CTLF levels are indicative of an improved neovascular response.

Table 15. Results of inhibition of ocular neovascularization in animals treated with gapmer compounds with choroidal neovascularisation (CNV) lesions.

TABLE 15
Results of inhibition of ocular neovascularization in animals treated
with gapmer compounds with choroidal neovascularisation (CNV) lesions.

| Treatment/SEQ ID NO: | % reduction | % reduction |
|---|---|---|
| (GAPMER COMPOUND NO.) | CTLF (7.8 μM) | CTLF (15 μM) |
| Saline | 0 | 0 |
| 224 (224) | 19 | 26 |

Gapmer compound 224 (SEQ ID NO 224) reduced CTLF in a dose-dependent manner.

Corneal neovascularization is a serious condition that can lead to a profound decline in vision. The abnormal vessels block light, cause corneal scarring, compromise visual acuity, and may lead to inflammation and edema. Corneal neovascularization occurs when the balance between angiogenic and antiangiogenic factors is tipped toward angiogenic molecules. Vascular endothelial growth factor (VEGF), one of the most important mediators of angiogenesis, is upregulated during neovascularization. Anti-VEGF agents have efficacy for neovascular age-related macular degeneration, diabetic retinopathy, macular edema, neovascular glaucoma, and other neovascular diseases. These same agents have great potential for the treatment of corneal neovascularization. Gapmer compound 224 was shown to reduce vascularization in response to choroidal neovascularisation (CNV) lesions.

We claim:

1. A gapmer compound comprising a modified oligonucleotide having 12 to 29 linked nucleosides in length, wherein the gapmer compound has a 5′ wing sequence having from about 3 to about 7 modified nucleosides, a central gap region sequence having from about 6 to about 15 2′-deoxynucleosides, and a 3′ wing sequence having from about 3 to about 7 modified nucleosides;
wherein the 5′ wing and 3′ wing modified nucleosides each comprise a sugar modification selected from the group consisting of a 2′-methoxyethyl (MOE) modification, a locked nucleic acid (LNA) modification, a 2′F-ANA modification, a 2′-O-methoxyethyl (2′OMe) modification, and combinations thereof;
wherein the linked nucleosides are linked with phosphorothioate internucleoside linkages, phosphorothiolate internucleoside linkages, or combinations thereof; and
wherein the modified oligonucleotide has a nucleobase sequence that is at least 91% complementary over its entire length to Region A nucleotides 256-282, Region B nucleotides 511-540, Region C nucleotides 523-547, Region D nucleotides 441-469, Region E nucleotides 88-107, or Region F nucleotides 547-567 of Upstream Master Lnc RNA Of The Inflammatory Chemokine Locus (UMLILO) long non-coding RNA SEQ ID NO: 231.

2. The gapmer compound of claim 1, wherein the gapmer compound has zero to one mismatch over its entire length to Region D nucleotides 441-469 of SEQ ID NO: 231.

3. The gapmer compound of claim 2, wherein the gapmer compound is at least 100% complementary over its entire length to Region D nucleotides 441-469 of SEQ ID NO: 231.

4. The gapmer compound of claim 1, wherein the gapmer compound has zero to one mismatch over its entire length to Region A nucleotides 256-282 of SEQ ID NO: 231.

5. The gapmer compound of claim 4, wherein the gapmer compound is at least 100% complementary over its entire length to Region A nucleotides 256-282 of SEQ ID NO: 231.

6. The gapmer compound of claim 1, wherein the gapmer compound has zero to one mismatch over its entire length to Region B nucleotides 511-540 of SEQ ID NO: 231.

7. The gapmer compound of claim 6, wherein the gapmer compound is at least 100% complementary over its entire length to Region B nucleotides 511-540 of SEQ ID NO: 231.

8. The gapmer compound of claim 1, wherein the gapmer compound has zero to one mismatch over its entire length to Region C nucleotides 523-547 of SEQ ID NO: 231.

9. The gapmer compound of claim 8, wherein the gapmer compound is at least 100% complementary over its entire length to Region C nucleotides 523-547 of SEQ ID NO: 231.

10. The gapmer compound of claim 1, wherein the gapmer compound has zero to one mismatch over its entire length to Region E nucleotides 88-107 of SEQ ID NO: 231.

11. The gapmer compound of claim 10, wherein the gapmer compound is at least 100% complementary over its entire length to Region E nucleotides 88-107 of SEQ ID NO: 231.

12. The gapmer compound of claim 1, wherein the gapmer compound has zero to one mismatch over its entire length to Region F nucleotides 547-567 of SEQ ID NO: 231.

13. The gapmer compound of claim 12, wherein the gapmer compound is at least 100% complementary over its entire length to Region F nucleotides 547-567 of SEQ ID NO: 231.

14. The gapmer compound of claim 1, selected from the group consisting of Gapmer Compound No. 223, 12, 21, 35-42, 55-56, 88, 100-102, 123-124, 127-128, 151-153, 155-162, 224-227, and 230.

15. The gapmer compound of claim 1, wherein the modified oligonucleotide is 18 linked nucleosides in length and has a nucleobase sequence consisting of the nucleobase sequence of SEQ ID NO: 223, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

16. The gapmer compound of claim 1, wherein the modified oligonucleotide is 18 linked nucleosides in length and has the nucleobase sequence consisting of the nucleobase sequence of SEQ ID NO: 224, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

17. The gapmer compound of claim 1, wherein the modified oligonucleotide is 16 linked nucleosides in length and has the nucleobase sequence consisting of the nucleobase sequence of SEQ ID NO: 225, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the 5′ wing segment consists of three locked nucleic acid (LNA) modified nucleosides; wherein the 3′ wing segment consists of three locked nucleic acid (LNA) modified nucleosides modified nucleosides; wherein each internucleoside linkage is a phosphorothioate linkage; and wherein each cytosine is a 5-methylcytosine.

18. The gapmer compound of claim 1, wherein the modified oligonucleotide is 16 linked nucleosides in length and has the nucleobase sequence consisting of the nucleobase sequence of SEQ ID NO: 226, wherein the modified oligonucleotide has a gap segment consisting of ten linked deoxynucleosides; a 5′ wing segment consisting of three locked nucleosides; and a 3′ wing segment consisting of three locked nucleosides; wherein the gap segment is positioned between the 5′ wing segment and the 3′ wing segment in the 5′ to 3′ direction; wherein the 5′ wing segment consists of three locked nucleic acid (LNA) modified nucleosides; wherein the 3′ wing segment consists of three locked nucleic acid (LNA) modified nucleosides modified nucleosides; wherein each internucleoside linkage is a phosphorothioate linkage; and wherein each cytosine is a 5-methylcytosine.

19. The gapmer compound of claim 1, wherein the modified oligonucleotide is 16 linked nucleosides in length and has the nucleobase sequence consisting of the nucleobase sequence of SEQ ID NO: 227, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the gap segment consists of nine deoxynucleosides and one 2′-O-methoxyethyl (2′-MOE) modified nucleoside at position 3 of the ten nucleosides starting from the 5′ position of the gap segment, the 5′ wing segment consists of three locked nucleic acid (LNA) modified nucleosides; wherein the 3′ wing segment consists of three locked nucleic acid (LNA) modified nucleosides modified nucleosides; wherein each internucleoside linkage is a phosphorothioate linkage; and wherein each cytosine is a 5-methylcytosine.

20. The gapmer compound of claim 1, wherein the modified oligonucleotide is 16 linked nucleosides in length and has the nucleobase sequence consisting of the nucleobase sequence of SEQ ID NO: 150, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the gap segment consists of ten deoxynucleosides, the 5′ wing segment consists of three locked nucleic acid (LNA) modified nucleosides; wherein the 3′ wing segment consists of three locked nucleic acid (LNA) modified nucleosides modified nucleosides; wherein each internucleoside linkage is a phosphorothioate linkage; and wherein each cytosine is a 5-methylcytosine.

21. A gapmer compound comprising a modified oligonucleotide having 12 to 29 linked nucleosides in length, wherein the modified oligonucleotide comprises a nucleobase sequence selected from the group consisting of SEQ ID NOs: 223, 12, 21, 35-42, 55-56, 88, 100-102, 123-124, 127-128, 151-153, 155-162, 224-227, and 230 wherein the gapmer compound has a 5′ wing sequence having from about 3 to about 7 modified nucleosides, a central gap region sequence having from about 6 to about 10 2′-deoxynucleosides, and a 3′ wing sequence having from about 3 to about 7 modified nucleosides,
wherein the 5′ wing and 3′ wing modified nucleosides each comprise a sugar modification selected from a 2′-methoxyethyl (MOE) modification, a locked nucleic acid (LNA) modification, a 2′F-ANA modification, a 2′-O-methoxyethyl (2′OMe) modification, or combinations thereof,
the linked nucleosides are linked with phosphorothioate internucleoside linkages, phosphorothiolate internucleoside linkages, or combinations thereof; and
wherein the modified oligonucleotide has a nucleobase sequence that is at least 91% complementary over its entire length to a nucleotide sequence of Upstream Master LncRNA Of The Inflammatory Chemokine Locus (UMLILO) long non-coding RNA wherein the UMLILO long non-coding RNA SEQ ID NO: 231.

22. The gapmer compound of claim 21, wherein the modified oligonucleotide is 18 linked nucleosides in length and has a nucleobase sequence consisting of the nucleobase sequence of SEQ ID NO: 223, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

23. The gapmer compound of claim 21, wherein the modified oligonucleotide is 18 linked nucleosides in length and has the nucleobase sequence consisting of the nucleobase sequence of SEQ ID NO: 224, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

24. A method for treating AMD or cytokine storm comprising administering to a subject, in need thereof, a therapeutically effective amount of a composition comprising a gapmer compound and a pharmaceutically acceptable excipient;
wherein the gapmer compound comprises a modified oligonucleotide having 12 to 29 linked nucleosides in length, wherein the gapmer compound has a 5′ wing sequence having from about 3 to about 7 modified nucleosides, a central gap region sequence having from about 6 to about 15 2′-deoxynucleosides, and a 3′ wing sequence having from about 3 to about 7 modified nucleosides;
wherein the 5′ wing and 3′ wing modified nucleosides each comprise a sugar modification selected from a 2′-methoxyethyl (MOE) modification, a locked nucleic acid (LNA) modification, a 2′F-ANA modification, a 2′-O-methoxyethyl (2′OMe) modification, or combinations thereof;
the gapmer compound linked nucleosides are linked with phosphorothioate internucleoside linkages, phosphorothiolate internucleoside linkages, or combinations thereof; and
wherein the modified oligonucleotide has a nucleobase sequence that is at least 91% complementary over its entire length to Region A nucleotides 256-282, Region B nucleotides 511-540, Region C nucleotides 523-547, Region D nucleotides 441-469, Region E nucleotides 88-107, or Region F nucleotides 547-567 of Upstream Master LncRNA Of The Inflammatory Chemokine Locus (UMLILO) long non-coding RNA SEQ ID NO: 231.

25. The method of claim 24, wherein the gapmer compound is selected from the group consisting of gapmer compound no. 223, 12, 21, 35-42, 55-56, 88, 100-102, 123-124, 127-128, 151-153, 155-162, 224-227, and 230.

26. The method of claim 24, wherein the gapmer compound is 18 linked nucleosides in length and has a nucleobase sequence consisting of the nucleobase sequence of SEQ ID NO: 223, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

27. The method of claim 24, wherein the gapmer compound is 18 linked nucleosides in length and has the nucleobase sequence consisting of the nucleobase sequence of SEQ ID NO: 224, wherein the modified oligonucleotide has a gap segment consisting of ten linked deoxynucleosides; a 5′ wing segment consisting of four linked nucleosides; and a 3′ wing segment consisting of four linked nucleosides; wherein the gap segment is positioned between the 5′ wing segment and the 3′ wing segment in the 5′ to 3′ direction; wherein the 5′ wing segment consists of four 2′-O-methoxyethyl (2′-MOE) modified nucleosides; wherein the 3′ wing segment consists of four 2′-O-methoxyethyl (2′-MOE) modified nucleosides; wherein each internucleoside linkage is a phosphorothioate linkage; and wherein each cytosine is a 5-methylcytosine.

28. The method of claim 24, wherein the gapmer compound is 16 linked nucleosides in length and has the nucleobase sequence consisting of the nucleobase sequence of SEQ ID NO: 230, wherein the modified oligonucleotide has a gap segment consisting of ten linked deoxynucleosides; a 5′ wing segment consisting of three linked nucleosides; and a 3′ wing segment consisting of three linked nucleosides; wherein the gap segment is positioned between the 5′ wing segment and the 3′ wing segment in the 5′ to 3′ direction; wherein the 5′ wing segment consists of three 2′F-ANA modified nucleosides; wherein the 3′ wing segment consists of three 2′F-ANA modified nucleosides; wherein each internucleoside linkage is a phosphorothioate linkage; and wherein each cytosine is a 5-methylcytosine.

29. The gapmer compound of any one of claims 1-23, wherein the locked nucleic acid modification is selected from a constrained ethyl (cEt) modification and a constrained methyl (cMe) modification.
